SEQUENCE LISTING (3 sequences)

SEQ ID NO: 1 - DNA, 34 bp, Artificial Sequence
  Synthetic
gtactcgcagtagtcagatgtgtataagagacag

SEQ ID NO: 2 - DNA, 42 bp, Artificial Sequence
  Synthetic
tacccttccaatttaaccctccaagatgtgtataagagacag

SEQ ID NO: 3 - DNA, 14 bp, Artificial Sequence
  Synthetic
  misc_feature (1)..(1): 5Phos
  misc_feature (14)..(14): ddC
ctgtctcttataca

(19) United States
(12) Patent Application Publication
(10) Pub. No.: US 2024/0254549 A1
(43) Pub. Date: Aug. 1, 2024

(54) PARTITIONING CELLS FOR HIGH THROUGHPUT SINGLE-CELL SEQUENCING

(71) Applicants: CHAN ZUCKERBERG BIOHUB, INC., San Francisco, CA (US); The Regents of the University of California, Oakland, CA (US)

(72) Inventors: Adam Abate, Oakland, CA (US); Xiangpeng Li, Oakland, CA (US); Benjamin Demaree, Oakland, CA (US)

(21) Appl. No.: 18/290,550

(22) PCT Filed: May 26, 2022
(86) PCT No.: PCT/US22/31149

§ 371 Date: Nov. 14, 2023

Related U.S. Application Data
(60) Provisional application No. 63/193,996, filed on May 27, 2021.

(51) Int. Cl. C12Q 1/6869; C12N 15/10

(52) U.S. Cl. CPC C12Q 1/6869; C12N 15/1065

(57) ABSTRACT

The present disclosure provides materials and methods for partitioning cells and high throughput, single-cell multi-omic sequencing. Methods for pathogen detection and identification, microbiome analysis, personalized medicine, environmental analysis where single-cell information is critical are each provided herein.

### CROSS-REFERENCE TO RELATED APPLICATIONS

This application claims the benefit of U.S. Provisional Application No. 63/193,996, filed May 27, 2021, the entire contents of which are fully incorporated herein by reference

### STATEMENT REGARDING FEDERALLY SPONSORED RESEARCH

The invention was made with Government support under Grant No. RO1 AI129206, awarded by The National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55876_Seqlisting.txt”, which was created on May 26, 2022 and is 989 bytes in size. The subject matter of the Sequence Listing is incorporated herein in its entirety by reference.

### FIELD

The present disclosure relates generally to methods for single-cell sequencing.

### BACKGROUND

Single-cell sequencing technologies refer to the methods to obtain genomics, transcriptomics or multi-omics information of single cells. Traditional sequencing methods only work with samples of many cells, and are thus unable to resolve cellular heterogeneity. Although several single-cell sequencing methods are available, there are many limitations. For example, microfluidics-based single-cell sequencing methods are technologically challenging for biologists to perform. Well plate-based methods lack sufficient throughput. As most available methods are targeted for transcriptome sequencing, single-cell genome sequencing and other multiomic technologies are not well established. There thus exists a need in the art for high throughput, single-cell sequencing.

### SUMMARY OF THE INVENTION

One embodiment of the present disclosure provides a method of sequencing one or more nucleic acids from a single cell comprising the steps of: (a) preparing single cells for coding comprising compartmentalizing a population of cells in permeable compartments comprising single cells; (b) barcoding nucleic acid molecules associated with the single cells, wherein a unique barcode is used for each single cell; and (c) sequencing one or more nucleic acids from a single cell.

In another embodiment, the compartmentalizing in step (a) comprises: (i) encapsulating single cells into hydrogel particles or (ii) fixating cells under conditions that allow formation of permeable, single-cell particles. In still another embodiment, the compartmentalizing comprises encapsulating single cells into hydrogel particles. In yet another embodiment, the preparing single cells for barcoding comprises: (a) preparing a suspension (e.g., in the gel or, in other embodiments, prior to gelation) comprising a population of single cells; (b) encapsulating the population of single cells in a polydispersed emulsion comprising gel droplets to provide a population of gel droplets, wherein each gel droplet contains zero cells, a single cell, or multiple cells; (c) polymerizing the population of gel droplets to provide a population of polymerized gel droplets; (d) separating the polymerized gel droplets by size under conditions that allow selection of a population of gel droplets that each comprise a single cell (and optionally where the conditions promote breaking the polymerized gel droplets); (c) optionally amplifying one or more biomolecules under conditions to increase copy number of said one or more biomolecules; and (f) lysing the single cells within the population of gel droplets under conditions that allow cell lysis.

In yet another embodiment, the steps of preparing a suspension in step (a) and encapsulating in step (b) are not performed using a microfluidic device. In another embodiment of the present disclosure, one or more of the steps of polymerizing in step (c), separating in step (d), optionally the amplifying in step (c), lysing in step (f), and barcoding are optionally not performed using a microfluidic device. In another embodiment, the preparing comprises the amplifying of step (c), and wherein said amplifying comprises an amplification technique selected from the group consisting of multiple displacement amplification (MDA), looping-based amplification cycles (MALBAC), degenerate oligonucleotide PCR (DOP-PCR) and primer extension pre-amplification (PEP). In still another embodiment, step (e) occurs before step (d).

In another embodiment, the present disclosure provides an aforementioned method wherein the suspension comprising the population of cells comprises an unpolymerized monomer solution. In still another embodiment, the unpolymerized monomer is selected from the group consisting of acrylamide, N,N′-Bis(acryloyl)cystamine (BAC), Bis(2-methacryloyl)oxyethyl disulfide (DSDMA), N,N′-(1,2-Dihydroxyethylene)bisacrylamide (DHEBA), N,N′-Methylene-bis-acrylamide or agarose. In yet another embodiment, the suspension further comprises a stain, antibody, aptamer, label, or affinity reagent capable of binding to one or more biomolecules including nucleic acids prior to and/or following cell lysis in step (e). In one embodiment, the stain is capable of binding to a cell membrane or cell wall.

In still another embodiment, the encapsulating the population of cells of step (b) comprises adding an immiscible carrier and agitating under conditions that allow formation of a polydispersed emulsion. In another embodiment, the agitating is selected from the group consisting of pipetting, shaking by hand, stirring, beating, bubbling, passing the solution through a needle or a narrow channel including, for example, a microfluidic channel within a microfluidic device, vortexing and sonicating.

In yet another embodiment of the present disclosure, the gel droplets are the approximate size of a mammalian cell. In various embodiments, the gel droplets are 4-20 um, 2-30 m, or 2-100 um.

In another embodiment, the polymerizing of step (c) comprises one or more of cooling, chemical crosslinking, photo-crosslinking, and ionic interaction crosslinking. In yet another embodiment, the separating of step (d) comprises one or more of centrifugation, density centrifugation, filtration, and/or collecting layers. In another embodiment, the lysing step (e) comprises adding a solution comprising a detergent and/or an enzyme and/or heating. In another embodiment, the detergent is selected from the group consisting of sodium dodecyl sulfate (SDS), Tween, Triton, Brij, Octyl glucoside, 3-[(3-Cholamidopropyl)dimethylammonio]-1-propanesulfonate (CHAPS), and 3-([3-Cholamidopropyl]dimethylammonio)-2-hydroxy-1-propanesulfonate (SHAPSO). In another embodiment, the enzyme is selected from the group consisting of lysozyme, proteinase K, lysostaphin, zymolyase, and mutanolysin.

The present disclosure also provides an aforementioned method wherein the lysing step (e) further comprises processing one or more biomolecules from the lysed cells, wherein said biomolecules comprise nucleic acids and/or proteins, and wherein said processing comprises digesting, labelling, capturing, and/or conjugating said biomolecules. In one embodiment, one or more biomolecules are labeled with a fluorophore or antibody. In another embodiment, one or more biomolecules are digested.

In yet another embodiment of the present disclosure, the compartmentalizing comprises fixating cells under conditions that allow formation of permeable, single-cell particles.

In another embodiment, the preparing single cells for barcoding comprises: (a) preparing a suspension comprising a population of single cells; (b) fixating single cells under conditions that allow formation of permeable, single-cell particles; (c) separating the single-cell particles; and (d) lysing the single cells within the single-cell particles under conditions that allow cell lysis. In another embodiment, the steps of preparing a suspension in step (a) and fixating in step (b) are not performed using a microfluidic device. In another embodiment, one or more of the separating in step (c), the lysing in step (d), and barcoding are optionally not performed using a microfluidic device. In another embodiment, step (d) occurs before step (c).

In yet another embodiment, the suspension further comprises a stain, antibody, aptamer, label, or affinity reagent capable of binding to one or more biomolecules including nucleic acids prior to and/or following cell lysis in step (d). In another embodiment, the stain is capable of binding to a cell membrane or cell wall. In another embodiment, the fixating single cells of step (b) comprises adding one or more fixation reagents selected from the group consisting of an organic solvent, a crosslinked fixative, or combinations thereof, under conditions that formation of permeable, single-cell particles. In another embodiment, the organic solvent is selected from the group consisting of methanol, ethanol, acetic acid, or combinations thereof. In another embodiment, the crosslinked fixative is selected from the group consisting of formaldehyde, paraformaldehyde, glutaraldehyde, acrolein, dithio-bis(succinimidyl propionate); oxidants: osmium tetroxide, potassium dichromate, or combinations thereof.

In still another embodiment, the separating of step (c) comprises one or more of centrifugation, density centrifugation, filtration, and/or collecting layers. In another embodiment, the lysing step (d) comprises adding a solution comprising a detergent and/or an enzyme and/or heating. In another embodiment, the detergent is selected from the group consisting of sodium dodecyl sulfate (SDS), Tween, Triton, Brij, Octyl glucoside, 3-[(3-Cholamidopropyl)dimethylammonio]-1-propanesulfonate (CHAPS), and 3-([3-Cholamidopropyl]dimethylammonio)-2-hydroxy-1-propanesulfonate (SHAPSO). In another embodiment, the enzyme is selected from the group consisting of lysozyme, proteinase K, lysostaphin, zymolyase, and mutanolysin.

In yet another embodiment, the lysing step (e) further comprises processing one or more biomolecules from the lysed cells, wherein said biomolecules comprise nucleic acids and/or proteins, and wherein said processing comprises digesting, labelling, capturing, and/or conjugating said biomolecules. In another embodiment, one or more biomolecules are labeled with a fluorophore or antibody. In another embodiment, one or more biomolecules are digested.

In still other embodiments, an aforementioned is provided wherein cells are selected from mammalian cells, bacterial cells, fungal cells, yeast cells, and plant cells. In another embodiment, the cells are bacterial cells. In another embodiment, the cells are human cells taken from a saliva, blood, urine, or tissue sample.

In yet other embodiments, an aforementioned method is provided wherein the one or more nucleic acids are selected from the group consisting of DNA, genomic DNA, RNA and mRNA.

In another embodiment, a method of determining the presence of a biomolecule associated (e.g., on a cell or within a cell) with a single cell is provided comprising the steps of preparing single cells for barcoding according to an aforementioned method, wherein one or more nucleic acids are conjugated to a reagent capable of specifically binding to the biomolecule.

### BRIEF DESCRIPTION OF THE DRAWINGS

FIG. 1 shows an example workflow of using hydrogel materials for partitioning cells for high throughput single cell sequencing. The cells were mixed with hydrogel material or monomers and emulsion were then generated. After gelation, the emulsions were broken and the hydrogel beads containing cells were generated. The hydrogel beads of the desired size range were then selected and the cell within the hydrogel beads were lysed. After washing, the single genomes trapped in the hydrogels were ready for single cell barcoding on commercial platforms.

FIG. 2 shows an example of hydrogel size selection. The droplets containing agarose hydrogel were generated by shaking the mixture of cell/agarose solution and oil (Left). The droplets with a size between 4-20 um were selected using buoyancy (Middle). After breaking the emulsion, the hydrogel was stained with SYBR green to show the cells (Right).

FIG. 3 shows an example of single-cell barcoding molecular biology workflow for DNA sequencing. Within droplets, the genomic DNA was tagmented with Tn5 to generate genome fragments with universal primer handles. The droplets were merged with barcoding beads and PCR regents, and thermocycled to produce amplicons with barcodes. Then the amplicons were purified and the sequencing adaptors were added by library PCR.

FIG. 4 shows the single cell genomic analysis of a sample consist of 4 microbiology (V. natriegens, P putida, B. subtilis, S. cerevisiae). Read count per barcode group plot (FIG. 4A) indicate the total number of cells passing filter. Histogram of barcode purity (FIG. 4B). Scatter plot of the percentage coverage of each barcode group with 95% purity vs the read counts (FIG. 4C). Cross talk between species in the barcode groups with 95% purity (FIG. 4D). Heatmap of read count across binned genome of all V natriegens barcode group indicate uniform coverage (FIG. 4E).

FIG. 5 shows an example of single cell barcoding molecular biology workflow for RNA sequencing. First, cDNA was synthesized by reverse transcription using random hexamer with a 5′ universal handle. Template switching was used to introduce a universal handle at the 3′ of cDNA. The cDNA was amplified with barcoding PCR. The sequencing library was then prepared by library PCR.

FIG. 6 shows an example of single cell RNA sequencing workflow design. The cells were fixed and permeabalized. Then the cDNA molecules were synthesized using by in cell reverse transcription reaction. The cells were then barcoded using a single cell commercial platform to generate single cell library.

FIG. 7 shows the schematics of high throughput single bacterial genomic DNA sequencing (EASI-seq). FIG. 7A: Microbiome cells suspension in hydrogel precursor and oil are mixed by passing through a syringe needle to form emulsions. After gelation, the cells are individually embedded in hydrogel beads. FIG. 7B: The hydrogel beads are size-selected using differential centrifugation. The hydrogel beads are suspended in a density matching buffer (40% sucrose in PBS with 0.1% tween20) and centrifuged. Particles of different sizes in the suspension sediment at different rates, with the larger particles sedimenting faster. After centrifuge at 1000×G for 10 min, the oversized hydrogel beads are pelleted, and the supernatants are subject to a centrifuge at a higher speed (3000×G for 10 min). The pellets are then collected as the size-selected hydrogel beads. The cells are then lysed by a 2-step enzyme digestion. The beads are first treated in 4 different enzymes that digest cell walls and then treated in protease to digest proteins. The small pore-size of hydrogel allows proteins and other molecules freely diffuse except long DNA molecules, after washing only genomic DNA retained in the hydrogel beads. FIG. 7C: The microbiome genomic DNA in hydrogel beads is tagmented in droplets and paired with barcode beads for barcoding PCR on a Mission Bio Tapestri instrument or custom-made microfluidics devices. FIG. 7D: Sequencing the amplicon generates barcoded single-cell shotgun reads for thousands of cells. FIG. 7E: EASI-seq allows high-throughput microbiome genome atlas analysis, as well as cluster-based genome assembly, strain identification, and pathway analysis.

FIG. 8 shows EASI-seq identifies single cells and has strain level resolution. FIG. 8A: ZymoBiomics microbial synthetic community (D6300, 10 species) was analyzed with EASI-seq for single cell resolution evaluation. FIG. 8B: The reads were filtered by read counts. FIG. 8C: Purity distribution of barcode groups, which is the percentage of the reads mapped to the majority species. The inset shows the purity distribution in log-scale. FIG. 8D: Representative read count barnyard plots. Each dot represents one barcode group and color-coded by species given in a) and mixture is labeled as grey. FIG. 8E: Coverages of each barcode group, color coded by species given in a). FIG. 8F: Average covered bases per reads for each cell type with color coding by species given in a). FIG. 8G: UMAP clustering by Taxonomic discovery algorithm, color coded by species given in a). Each barcode group is classified using a k-mer based taxonomy classifier (Kraken2). The output files were combined at genus level. The barcodes were filtered by the percentage of mapped reads and taxonomical purity, which is the percentage of the dominant taxa. The vector of the genus abundance in each barcode was used to generate the UMAP and each barcode is annotated by the most abundant genus. The inset UMAP shows the integration of the EASI-seq data (gray) and metagenomic data (blue). Each contig in the assembled metagenome of the same sample was treated as a barcode and processed by the Taxonomic discovery algorithm. The metagenomic contigs were integrated by using the Scanpy ingest tool module. FIG. 8H: The barcode and read counts in each cluster, grouped by batch (SiC-Seq or Metagenome assembly). FIG. 8I: Evaluation of assembled contigs by grouped reads from all barcodes in each cluster (left: Genome coverage, middle: relative contig length normalized to reference genome, and right: error rate in the cluster). All the reads within a cluster were assembled into contigs by Spades. The contigs were evaluated by Quast with reference genome. The error rates were calculated as the percentage of reads in each cluster that not mapped to reference genome (Bowtie2). FIG. 8J: Phylogenetic tree of the synthetic community consists of 22 equally mixed E. lenta used to evaluate the strain resolution of EASI-seq. FIG. 8K: Heatmap of strain abundance in each barcode group, grouped by identified strain (color-coded by strain given in j, and mixed/unresolved barcode is labeled in grey). The reads in each barcode are mapped to the 22 reference genomes and the abundances of each strain in the barcodes were calculated by Bayesian estimation. FIG. 8L: UMAP clustering based on Bayesian abundance estimation of 22 E. lenta strains in each barcode group, color-coded by strain given in j, and mixed/unresolved barcode is labeled in grey. The inset pie plot showed the barcode counts of each strain (excluding the mixed or unresolved barcodes).

FIG. 9 shows EASI-seq analysis of human fecal microbiome. FIG. 9A: Integrated UMAP clustering of the single cell barcodes and metagenomic assembled contigs of a human microbiome sample. Each barcode/contig was annotated by most abundant genus (matching the color code of species given in b). UMAP clustering highlights the data batch (single cell in red, and metagenomic contigs in blue). FIG. 9B: The comparison between metagenomic and single cell sequencing. The scatter plot shows the relative abundance of genus found by Kraken2 in metagenomics sequencing data and combined barcodes groups data (includes all barcodes before filtering). All genus discovered by the taxonomic discovery algorithm are shown in circles and scaled by barcode counts, the rest are shown in triangles. Top 30 clusters are listed in the legend. FIG. 9C: The barcode and read counts in each cluster, grouped by batch (SiC-Seq or Metagenome assembly). FIG. 9D: Sub Clustering of EASI-seq barcodes in the top 3 clusters (Blautia_A, Bifidobacterum, and Collinsella) by species abundance estimation. FIG. 9E: Relative pathway abundance in the identified clusters. All reads in EASI-seq barcode groups associated to each cluster were combined and analyzed using MetaPhlAn with MetaCyc database. The relative abundances of each pathway (copy per million, CPMs) were normalized to the barcode counts in each cluster.

FIG. 10 shows single cell analysis of environmental microbiome (Ocean Beach sea water). FIG. 10A: UMAP generated by Kraken2 assigned genera color-code with most abundance genus in each barcode group. FIG. 10B: Barcode count distribution of each cluster. FIG. 10C: Read count distribution of each cluster. FIG. 10D: Venn diagram representing the number of clusters that were identified with antibiotic resistant genes (ARG), common phage sequences, and plasmids. FIG. 10E: Distribution of antibiotic resistant genes (ARG), phage sequences, plasmids and virulence factors in the 876 identified clusters. FIG. 10F: Relative potential for transduction between the clusters identified by single cell taxonomic discovery, determined by the relative number of common phage sequences detected in their respective genomes, plotted as a heat map. FIG. 10G: Distribution of antibiotic resistant genes in the identified clusters.

### DETAILED DESCRIPTION

The present disclosure provides methods and compositions for high throughput single-cell multi-omic sequencing that is simple to operate. The present disclosure provides a rapid method of high-throughput, single-cell sequencing using single-cell partitioning techniques described herein. Methods for pathogen detection and identification, microbiome analysis, personalized medicine, environmental analysis where single-cell information is critical are each provided herein.

The methods and compositions provided herein provide several key innovations over existing technologies. First, in one embodiment of the disclosure, single cells are isolated and encapsulated in hydrogel microbeads, by shake emulsification. The hydrogel microbeads are size-selected based on buoyancy and centrifugation force. This embodiment allows for fast processing of millions of cells without any complex instrumentation such as microfluidics or fluorescence-activated cell sorting (FACS) and at a throughput surpassing other available methods.

Second, in another embodiment, the single cells embedded with in hydrogel microbeads are lysed and washed in solution. Because the hydrogel materials allow free diffusion of any molecules with hydraulic diameters smaller than the pore size, but sterically trap genomic DNA, this invention allows multi-step molecular biology reactions required for genomic sequencing which are not easily performed in other systems. Also, the existing single-cell analysis platform such as microwell, microbeads, or microfluidic-based barcoding methods lack the ability to perform multi-step reactions or the workflows are long and challenging to perform.

Third, in still another embodiment, the present disclosure enables single-cell multi-omics sequencing. Before encapsulation or prior to cell lysis, cells can be stained with aptamers, DNA sequence-tagged antibodies or other antigen-binding molecules, and the DNA tag or aptamer sequences are read out by next generation sequencing. This provides a protein epitope profile of individual cells. Also, in another embodiment Poly-T capture sequence or template switching oligonucleotides can be used to capture mRNA, which provide transcriptome information from single cells.

As described herein, another embodiment of the present disclosure provides partitioned cells by fixating single cells.

Among the numerous improvements and advantages provide herein, the present disclosure provides methods that require minimal instrumentation, significantly lowering the technical expertise required to deploy single-cell whole genome sequencing, single-cell sequencing of bacteria and fungi on commercial platforms designed for mammalian cells, and multi-omic single-cell sequencing including genomics, transcriptomics, and proteomics.

### Definitions

As used herein, the term “sample” or “biological sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples. “Sample” and “biological sample” includes cells, e.g., bacterial cells or eukaryotic cells; biological fluids such as blood, cerebrospinal fluid, semen, saliva, and the like; bile; bone marrow; skin (e.g., skin biopsy); and viruses or viral particles obtained from an individual.

As described more fully herein, in various aspects the subject methods may be used to detect and/or quantify a variety of components from such biological samples. Components of interest include, but are not necessarily limited to, cells (e.g., circulating cells and/or circulating tumor cells), viruses and viral genomes, polynucleotides (e.g., DNA and/or RNA), polypeptides (e.g., peptides and/or proteins), and many other components that may be present in a biological sample. As described herein, the present disclosure provides methods and compositions for detecting and quantitating materials from single cells.

The terms “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 109 nucleotides or larger. Polynucleotides and nucleic acids include RNA, cDNA, genomic DNA. In particular, the polynucleotides and nucleic acids of the present invention refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al. Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

### Methods

The present disclosure provides methods and materials for sequencing one or more nucleic acids from a single cell. The methods provided herein comprising encapsulating cells in permeable compartments without microfluidic control. In some embodiments, the permeable compartments are achieved by (1) encapsulating the cells in hydrogel microbeads, or (2) fixation and permeabilization of the cells. Single cells are then barcoded using methods and compositions provided herein.

### 1. Hydrogel-Based Compartmentalization

Hydrogel-based compartmentalization comprises, in some embodiments, mixing cells with gel precursor materials, adding an immiscible carrier, and agitating the mixture. The agitation can comprise passing the fluids through a constriction, such as a syringe needle or microchannel network, or by shaking the mixture in a reservoir, such as with a vortexer, homogenizer, or shaking the tube. The resultant emulsion will comprise a range of droplet sizes, some of which contain single cells. The loading rate of the cells can be controlled by adjusting cell concentration, dilution, and addition of precursor materials prior to agitation. Particle properties can be selected to facilitate this, for example, by controlling particle chemistry, porosity, and functionalization. Once encapsulated, the sample is solidified to produce particles. These particles can comprise hydrogels, polymers, plastics, glasses, etc. The resultant particles can be further processed to enable single cell sequencing, including particle size selection and cell analyte preparation. These steps can be done in any order optimal for the particular workflow. To facilitate processing, particles can be transferred between carrier phases using a number of techniques, such as chemical or electrical demulsification, solvent transfer, particle templated emulsification, etc.

To facilitate later steps involving barcoding, permeable compartments of optimal size can be selected from the polydisperse suspension. In some embodiments, this is achieved by filtering the suspension with a series of filters to select a desired size range. Alternatively, particles can be selected based on filtering or density gradient centrifugation, collecting or discarding appropriate layers. In general, particles of a size similar to mammalian cells are optimal for barcoding with instruments designed for mammalian cell sequencing. If other instruments are to be used that are designed for barcoding different samples, a different size particle can be selected, as optimal for the workflow. Other methods for selecting particles contemplated by the present disclosure involve the use of hydrodynamic forces, some of which involve microfluidics. For example pinched flow fractionation and inertial ordering are passive techniques for selecting desired particles. Flow cytometry, an active sorting technique, may also be used to select particles based on optical properties. This provides additional benefits, such as allowing cell contents to be analyzed and used to inform selection.

To facilitate access to cell-based analytes, such as nucleic acids, the cells encapsulated in the particles can be processed, for example to lyse cell walls or membranes, capture mRNA or proteins, and the like. In some embodiments, this step can be achieved with the particles in an immiscible (e.g. oil) or miscible (e.g. aqueous) carrier to facilitate transfer of necessary materials into and out of the particles. Reagents can be mixed with the particles to prepare cells and their biomolecules for analysis. For example, detergents, enzymes (e.g. lysozyme, proteinase K), can digest cell molecules to afford access to nucleic acids and digest molecules that could interfere with later steps, such as nucleases. In the case of eukaryotic cells, chromatin may be digested to facilitate access to genomic DNA. Other digestions can also be performed to facilitate analysis. For example, nuclease digestion can be used to fragment genomic DNA into pieces suitable for sequencing. Tagmentation can be used to fragment and add universal adaptors for barcoding and/or sequencing. Alternatively, cellular analytes can be amplified to facilitate their analysis. For example, genomic DNA from single cells can be subjected to whole genome amplification to provide multiple copies for later analysis which, according to some embodiments of the present disclosure, increases the comprehensiveness and quality of the data obtained by the present methods. Upon lysis and/or digestion of cells and their biomolecules, the embedding particle matrix can facilitate capture of desired biomolecules. For example, in some embodiments polyT oligos attached to the particle backbone may capture released mRNA, or affinity molecules, like aptamers or antibodies, may capture specific epitopes released from cells. The particle properties, such as porosity, may capture molecules larger than a certain size, such as macromolecular DNA that may be sterically trapped within the particle.

In still other embodiments, cell-containing particles can be further processed to label them and their contents. For example, in some embodiments antibodies may bind to specific cells encapsulated in the particles, or fluorescent oligos may hybridize to cellular nucleic acids, such as mRNA, captured in the particles. These labels may facilitate later analysis according to the present disclosure, for example, making specific particles fluorescent for targeted recovery, or providing additional sequences by which to attach barcodes or other useful adaptors for sequencing. Labeled or unlabeled particles may be subjected to further processing, such as activated sorting by FACS or MACS. Alternatively, passive selection may also be performed, for example, by adding to processed particles a chemical that permits specific particles to survive while melting others based on their contents.

### 2. Fixed Cell as Compartmentalization

As provided herein, fixation and permeabilization based compartmentalization of cells comprises in various embodiments crosslinking fixative, organic solvent, or oxidants. The fixed cells can be permeabilized by treatment with organic solvents, surfactants, or enzymes according to some embodiments of the present disclosure.

To facilitate access to cell based analytes, such as nucleic acids, the fixed and permeabilized cells can be processed. For example, in some embodiments the cells can be processed to reverse transcribed to convert mRNA to cDNA, etc. Optionally, this step can be combined with template switching, ligation, or tagmentation to attach universal adaptors for barcoding and/or sequencing. In some embodiments, genomic DNA can also be tagmented into pieces suitable for sequencing. Tagmentation can be used to fragment and add universal adaptors for barcoding and/or sequencing. Alternatively, in some embodiments cellular analytes can be amplified to facilitate their analysis. For example, genomic DNA from single cells can be subjected to whole genome amplification to provide multiple copies for later analysis. This could, in some cases, increase the comprehensiveness and quality of the data as provided herein. Biomolecules other than nucleic acids can also be analyzed by staining prior to or after fixation and permeabilization steps in other embodiments. For example, in some embodiments affinity molecules, like aptamers or antibodies, may capture specific epitopes released from cells. These labels may facilitate later analysis, for example, making specific particles fluorescent for targeted recovery, or providing additional sequences by which to attach barcodes or other useful adaptors for sequencing. Labeled or unlabeled particles may be subjected to further processing, such as activated sorting by FACS or MACS.

### 3. Barcoding

The processed hydrogels or fixated cells provided herein are, according to some embodiments of the present disclosure, subjected to barcoding to enable scalable single cell sequencing. This can be accomplished with or without microfluidics using a variety of techniques. For example, in some embodiments with microfluidics, single step workflows can be used in which processed particles or cells contain cellular analytes that can be readily barcoded in a single step. For example, processed hydrogels or cells can be introduced into a microfluidic device that randomly pairs them with barcode sequences, such that the barcode sequences are incorporated into the processed analytes, permitting detection by a sequencing instrument. Alternatively, in other embodiments, microwell techniques that function along similar principles can perform this step. This step can also be accomplished using non-microfluidic techniques. For example, in some embodiments processed particles or cells can be subjected to split pool workflows that randomly attach barcodes using a combination of molecular techniques, such as tagmentation, ligation, and polymerase extension. Particle templated emulsification may also be used to randomly pair cell particles with barcodes.

The material resulting from the aforementioned processing and barcoding steps can then be analyzed, using for example sequencing, mass spectrometry, imaging, or other methods known in the art. The barcode information can be used to computationally group together all analytes (e.g., nucleic acids) originating from a single particle, thereby aggregating together information from single cells encapsulated in the particles, and multiple cells such as in paired cell studies.

Thus, as described herein, a method for sequencing single cells that use hydrogel-based permeable compartments for partitioning single cells comprises, in various embodiments, one or more of the following steps:
1. Processing cells to prepare them for barcoding
   a. Controlling concentration
   b. Labeling with stains, nucleic acids, aptamers, antibodies, DNA labeled affinity reagents, and the like.
2. Without microfluidic control, encapsulating cells in polydispersed particles
   a. Needle, vortex, shaking microfluidic drop generation
   b. Controlling the particle properties (e.g., chemistry, porosity, functionalization) to facilitate later processing.
3. Processing the particles to select an optimal size
   a. While in water or oil
   b. Density centrifugation, filtration, etc.
   c. Active or passive (inertial, pinched flow) sorting
4. Preparing cell based analytes (proteins, nucleic acids) for analysis
   a. In water or oil
   b. With or without microfluidics
   c. Adding reagents to lyse cells while retaining desired biomolecules
   d. Digesting biomolecules (e.g. proteins)
      i. Chromatin, inhibitors
      ii. Fragmentation of genomic DNA
         1. Tagmentation
   e. Capturing analytes
      i. Steric capture of genomic DNA
      ii. Targeted capture of mRNA or proteins
      iii. Attaching adapters
         1. Ligation, tagmentation, to mRNA, DNA, or genomic DNA
   f. Amplifying analytes
      i. e.g. MDA applied to genome to provide multiple copies
   g. Labeling captured analytes with detection components
      i. DNA-conjugated affinity reagents, aptamers, DNA binding chemicals, mRNA probes, etc.
   h. If necessary, further selecting particles based on labeled analytes
      i. MACS, FACS, etc.
      ii. Chemicals that melt gels either containing or lacking specific analytes.
5. Barcoding processed analytes to enable analysis
   a. Microfluidics
      i. 1 step workflow
         1. 10× Chromium (10× Genomics), SeqWell (Aicher et al., Methods. Mol. Biol. (2019) 1979:111-132)
      ii. 2 step workflow
         1. MissionBio Tapestri platform (Mission Bio)
   b. Non microfluidics (split pool, Fluent)
      i. Parse Biosciences Split pool based barcoding (Parse Biosciences; Rosenburg et al., Science (2018) 360:6385, 176-182)
      ii. Fluent Biosciences PIPseq (Fluent Bioscience; Hatori, M., et al., Anal. Chem. (2018) 90, 16, 9813-9820)
6. The resultant material recovered and prepared for analysis
   a. Sequencing, mass spectrometry, imaging, etc.
7. Where steps 3 and 4 may be done in reverse order

Additionally, as described herein, a method for sequencing single cells that fixed cells as permeable compartments for partitioning single cells comprises, in various embodiments, one or more of the following steps:
1. Processing cells to prepare them for barcoding
   a. Controlling concentration
   b. Labeling with stains, nucleic acids, aptamers, antibodies, DNA labeled affinity reagents, etc.
2. Cell fixation and permeabilization
   c. fixation reagents: organic solvents such as methanol, ethanol, acetic acid or their mixtures; crosslinked fixative: formaldehyde, paraformaldehyde, glutaraldehyde, acrolein, dithio-bis(succinimidyl propionate); oxidants: osmium tetroxide, potassium dichromate, etc. . . . .
   d. Organic solvent, surfactant and enzymes for permeabilization.
3. Preparing cell based analytes (proteins, nucleic acids) for analysis
   e. With or without microfluidics
   f. Adding reagents to lyse cells while retaining desired biomolecules
   g. preparing the nucleic acid with universal priming adaptors
      i. Reverse transcription for RNA
         1. Template switching
         2. ligation
         3. tagmentation
      ii. Fragmentation of genomic DNA
         1. Tagmentation
   h. Amplifying analytes
      i. e.g MDA applied to genome to provide multiple copies
   i. Labeling captured analytes with detection components
      i. DNA-conjugated affinity reagents, aptamers, DNA binding chemicals, mRNA probes, etc.
   j. If necessary, further selecting particles based on labelled analytes
      i. MACS, FACS, etc.
4. Barcoding processed analytes to enable analysis
   i. Microfluidics 1 step workflow
      1. 10× Chromium (10× Genomics), SeqWell (Aicher et al., Methods. Mol. Biol. (2019) 1979:111-132)
   ii. 2 step workflow
      1. MissionBio Tapestri platform (Mission Bio)
   b. Non microfluidics (split pool, Fluent)
   i. Parse Biosciences Split pool based barcoding (Parse Biosciences; Rosenburg et al., Science (2018) 360:6385, 176-182)
   c. Fluent Biosciences PIPseq (Fluent Bioscience; Hatori, M., et al., Anal. Chem. (2018) 90, 16, 9813-9820)
5. The resultant material recovered and prepared for analysis
   Sequencing, mass spectrometry, imaging, etc.

In some embodiments, detection of one or more biomolecules is also contemplated. “Detecting” as used herein generally means identifying the presence of a target, such as a target nucleic acid or protein. In various embodiments, detection signals are produced by the methods described herein, and such detections signals may be optical signals which may include but are not limited to, colorimetric changes, fluorescence, turbidity, and luminescence. Detecting, in still other embodiments, also means quantifying a detection signal, and the quantifiable signal may include, but is not limited to, transcript number, amplicon number, protein number, and number of metabolic molecules. In this way, sequencing or bioanalyzers can be employed in certain embodiments.

As described herein, some methods of the present disclosure include particles that provide emulsions. As described herein, particles include, but are not limited to, hydrogel beads, plastic beads, glass beads, ceramic beads, and magnetic beads. In certain embodiments, the hydrogel is selected from naturally derived materials, synthetically derived materials and combinations thereof. Examples of hydrogels include, but are not limited to, collagen, hyaluronan, chitosan, fibrin, gelatin, alginate, agarose, chondroitin sulfate, polyacrylamide, polyethylene glycol (PEG), polyvinyl alcohol (PVA), polyacrylamide/poly(acrylic acid) (PAA), hydroxyethyl methacrylate (HEMA), poly N-isopropyl acrylamide (NIP AM), and polyanhydrides, polypropylene fumarate) (PPF).

In related embodiments, as described herein a population of cells are prepared in a suspension in an unpolymerized monomer solution. In some embodiments, the unpolymerized monomer is selected from the group consisting of acrylamide, N,N′-Bis(acryloyl)cystamine (BAC), Bis(2-methacryloyl)oxyethyl disulfide (DSDMA), N,N′-(1,2-Dihydroxyethylene) bisacrylamide (DHEBA), N,N′-Methylene-bis-acrylamide or agarose.

According to some embodiments of the present disclosure, a lysing reagent is used in the detection methods. Lysing agents may include, for example chemical lysis, such as SDS, detergents, alkaline, and acid; biological lysis, such as lysis enzymes, viruses, and phages; and physical lysis such as beads beating, grinding. frozen-thaw, and sonication.

The present disclosure provides methods of detecting a target in a sample, or sequencing a nucleic acid where the sample is a single cell, where the target may be, for example, a nucleic acid (RNA, DNA), biomolecules such nucleic acids, genes, proteins or polypeptides or epitopes, as well as biological particles such as cells (bacterial, human, parasite) and viruses.

Exemplary pathogenic bacteria or bacterial cells include, for example, members of the genus Actinomyces, Bacillus, Bacteroides, Bordetella, Bartonella, Borrelia (e.g., B. burgdorferi OspA), Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus polypeptides, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus polypeptides (i.e., S. pneumoniae polypeptides), Proteus, Pseudomonas, Rickettsia, Rochalimaea, Salmonella, Shigella, Staphylococcus, group A streptococcus (e.g., S. pyogenes), group B streptococcus (S. agalactiae), Treponema, and Yersinia.

Exemplary pathogenic viruses or virus particles or viral genomes include, for example, adenovirus, alphavirus, calicivirus (e.g., a calicivirus capsid antigen), coronavirus polypeptides, distemper virus, Ebola virus polypeptides, enterovirus, flavivirus, hepatitis virus (AE), herpesvirus, infectious peritonitis virus, leukemia virus, Marburg virus, orthomyxovirus, papilloma virus, parainfluenza virus, paramyxovirus, parvovirus, pestivirus, picorna virus (e.g., a poliovirus), pox virus (e.g., a vaccinia virus), rabies virus, reovirus, retrovirus, and rotavirus. In certain embodiments, the virus is SARS-CoV-2, HIV, HSV, or HPV.

Exemplary parasites include protozoan parasites, for example, members of the Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Examples of helminth parasites include, but are not limited to, Acanthocheilonema, Aclurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca Spirometra, Stephanofilaria, Strongyloides, Strongylus, Thelazia, Toxascaris, Toxocara, Trichinella, Trichostrongylus, Trichuris, Uncinaria, and Wuchereria. Pneumocystis, Sarcocystis, Schistosoma, Theileria. Toxoplasma, and Trypanosoma are also contemplated.

Suitable subjects for the methods disclosed herein include mammals, e.g., humans. The subject may be one that exhibits clinical presentations of a disease condition, or has been diagnosed with a disease. In certain aspects, the subject may be one that has been diagnosed with an infection, e.g., COVID-19, or cancer, exhibits clinical presentations of infection or cancer.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### Example 1

### Whole Genome Sequencing Using the Hydrogel Based Compartmentalization

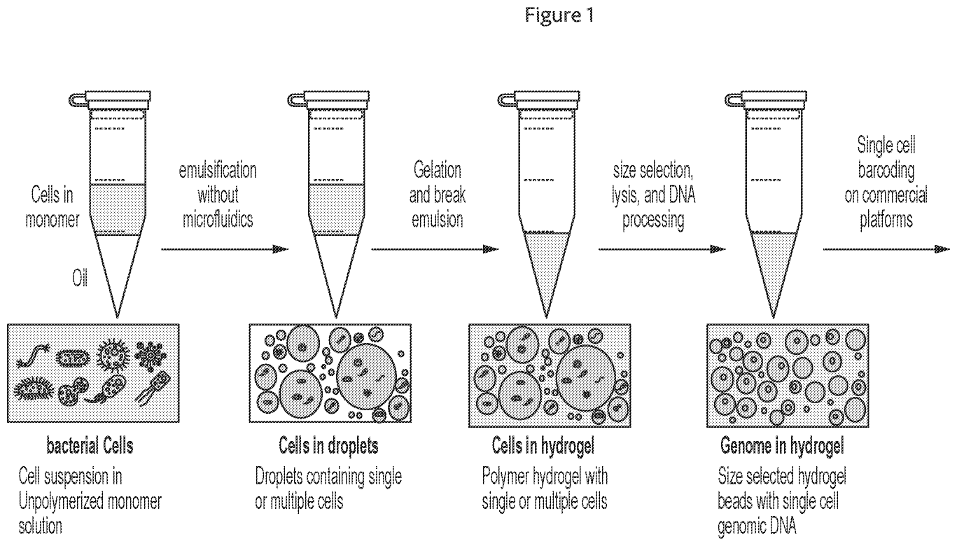

The following Example provides a workflow for single-cell sequencing using hydrogel-based compartmentalization. FIG. 1 also provides the general process for single-cell sequencing according to one embodiments of the disclosure.

### Workflow for Cell Encapsulation:

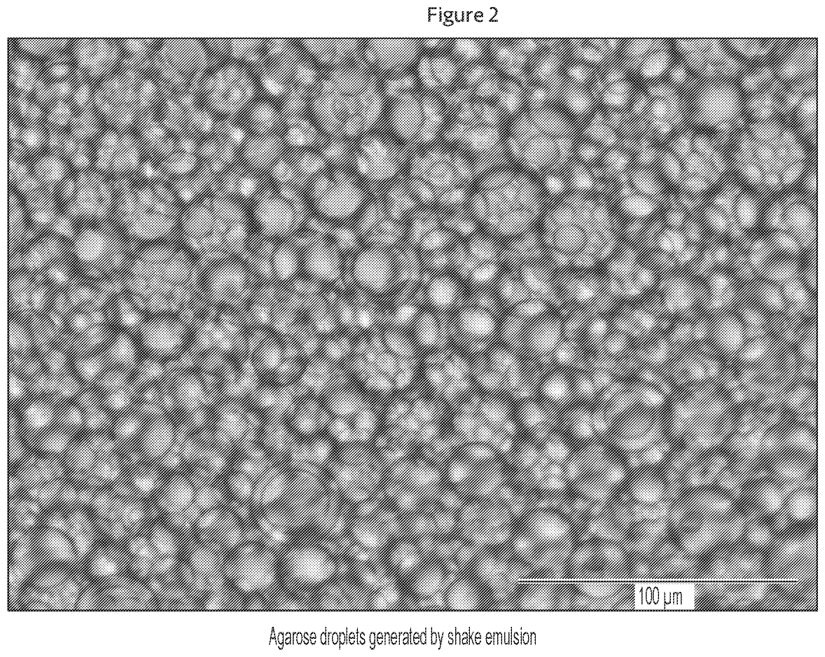

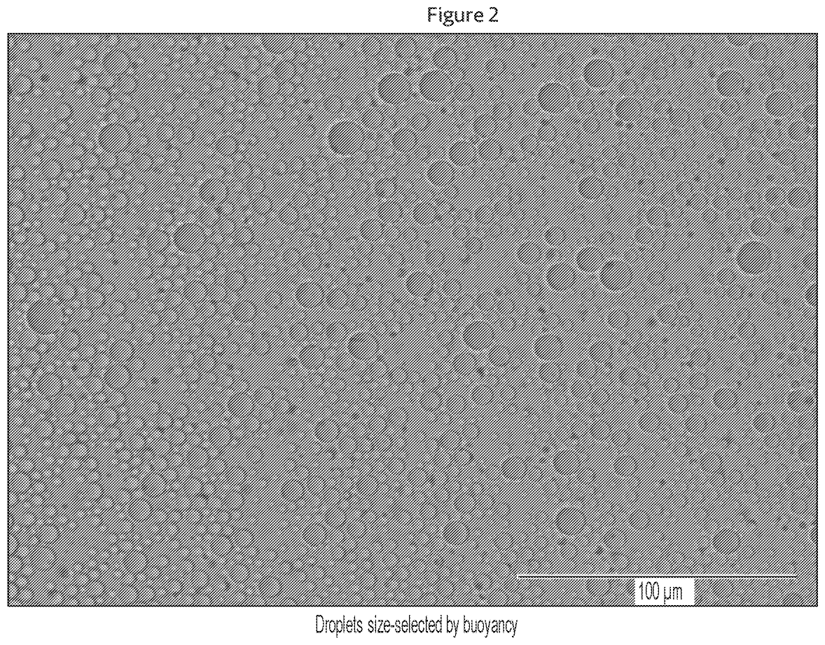

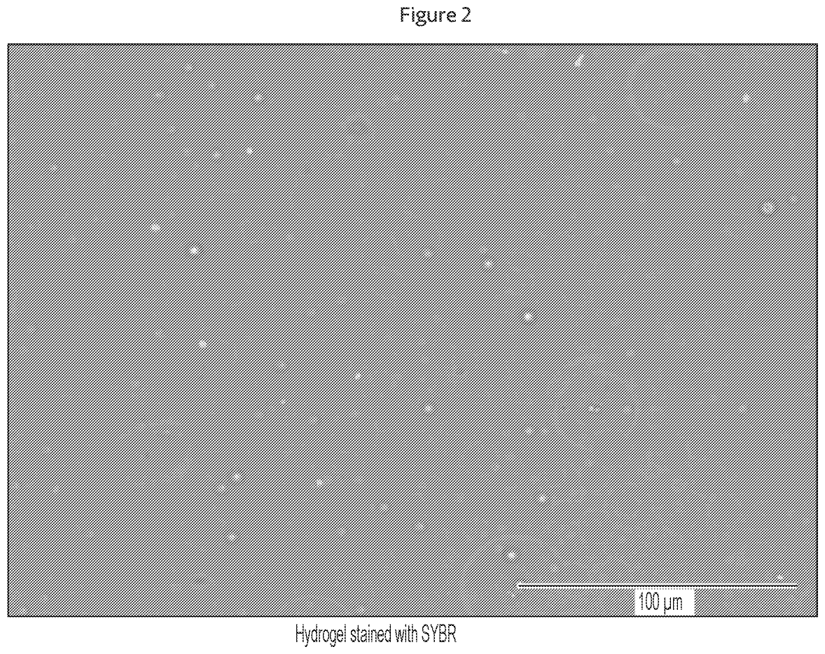

1. Collect cell sample.
2. Create single-cell suspension at correct concentration.
3. Mix cells with acrylamide monomer.
4. Add oil layer 1:1 (1 mL oil+1 mL cells/monomer)
5. Use syringe to make emulsions. Pass emulsion through syringe multiple times. (FIG. 2)
6. Allow emulsion to settle 10 m. Remove top layer of emulsion to remove large drops. (FIG. 2)
7. Add TEMED initiator (10 uL into emulsion). 70 C 20 m or RT overnight.
8. Break emulsion.
   a. Spin droplets, remove bottom oil layer.
   b. Add 20% PFO in HFE.
   c. Incubate.
9. Wash broken emulsions twice in PBS+Tween. (FIG. 2)
10. Perform sucrose size selection. (FIG. 2)
   a. Spin 10 m 500 g—keep supernatant
   b. Spin 10 m 1000 g—keep supernatant
   c. Spin 10 m 3000 g—collect pellet (contains size selected beads [5-15 um]).
11. Wash selected beads to remove sucrose.
   a. Wash 3× in PBS (4000 g 5 m)
12. Perform cell lysis in gels.
   a. Lysis cocktail—same as SiC-seq but no DTT [lysozyme, zymolyase, mutanolysin, lysostaphin]
13. Wash once with PBS, then add Proteinase K to digest protein.
14. Wash twice with PBS, soak in 100% ethanol 20 m to deactivate PK.
15. Wash 5× in PBS.
16. Resuspend beads to correct concentration for encapsulation on Tapestri.

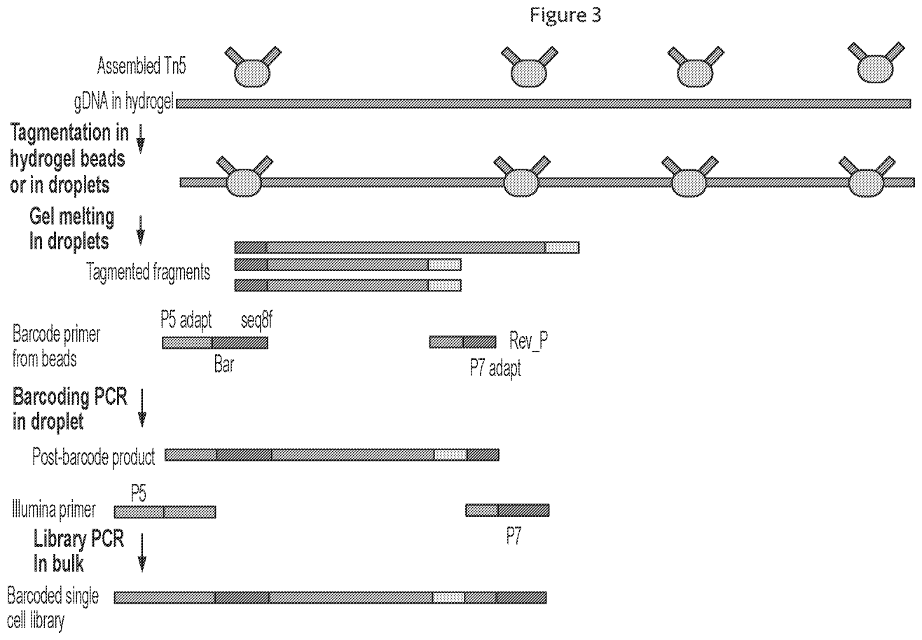

### Workflow for Tagmentation and Barcoding (FIG. 3):

1. Prepare tagmentation buffer with Tn5 and 10 mM DTT.
2. Co-flow tagmentation buffer with gel beads.
3. Incubate 37 C 1 hr, 50 C 20 m. Can be optimized.
4. Perform barcoding on Tapestri:
   a. Components: Mission Bio barcoding mix, reverse primer [0.8 uM final concentration in droplet]
   b. Run barcoding program on Tapestri
   c. UV treat and barcoding PCR (proceed with standard MB library prep)
   d. Break the emulsion and purify the barcoded oligo.
   e. Library PCR to generate the sequencing library

### Workflow for Library Sequencing and Data Analysis (FIG. 3):

1. The library is sequenced on Illumina MiSeq, NextSeq, HiSeq or NovaSeq platform with paired end setup.
2. The quality of the reads in raw fastq files were analyzed using Fastqc.
3. The barcode in the read files were extracted and the files were spitted according to the barcode sequences.
4. Each individual files were analyzed using Bowtie2, Samtools and custom written scripts.

### Example 2

### Single-Cell RNA Sequencing Using Fixed Cells as Compartmentalization

The following Example provides a workflow for single-cell sequencing using fixed cell compartmentalization.

### Workflow for in Bulk Cell Processing:

1. Collect cell sample.
2. Fix the cells with formaldehyde
3. Wash the cells to remove extra formaldehyde
4. Cell wall digestion with detergents, lysozyme and other enzymes.
5. Wash the cells to remove extra enzymes.
6. In-cell cDNA synthesis.
   a. in cell RNA polyadenylation using E coli poly(A) polymerase.
   b. Reverse transcription using poly(T) primer with 5′ PCR handle for barcoding.
   c. in-cell template switch reaction to introduce the 3′ handle for barcoding PCR
   d. alternatively, if a random hexamer primer is used in step b, step a can be skipped.
   e. alternatively, step c can replaced by 3′ labeling reaction using terminal transferase to introduce the 3′ handle.
7. Wash the cells to remove extra enzyme and primers.

### Workflow for Lysis and Barcoding:

1. Generate droplets in co-flow device with cell lysis buffer and cell suspension.
2. Incubate at 50 C for 50 min, 80 C for 10 min.
3. Perform barcoding on Tapestri:
   a. Components: Mission Bio barcoding mix, reverse primer (0.8 uM final in droplet)
   b. Run barcoding program on Tapestri
   c. UV treat and thermal cycling
4. Library preparation

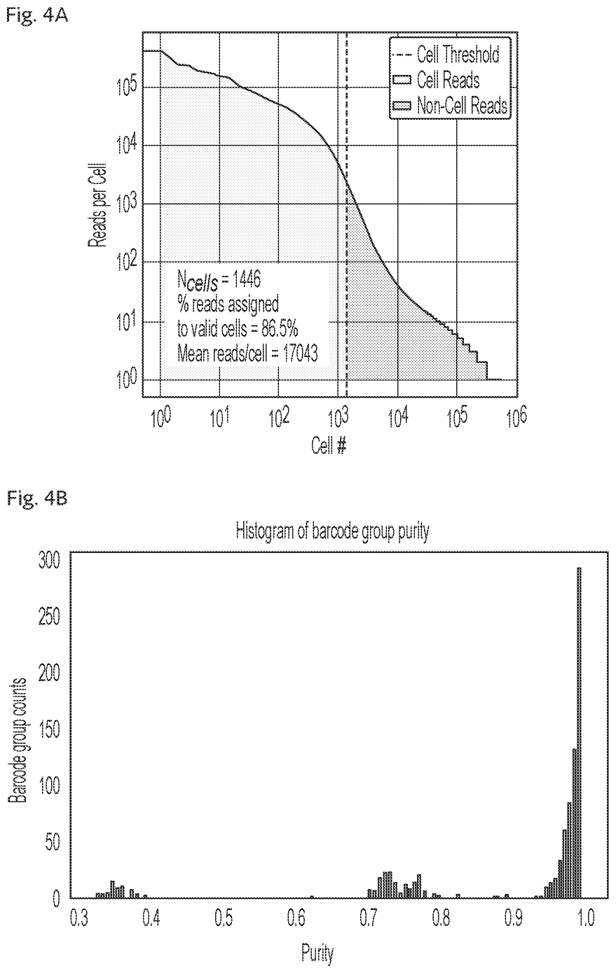

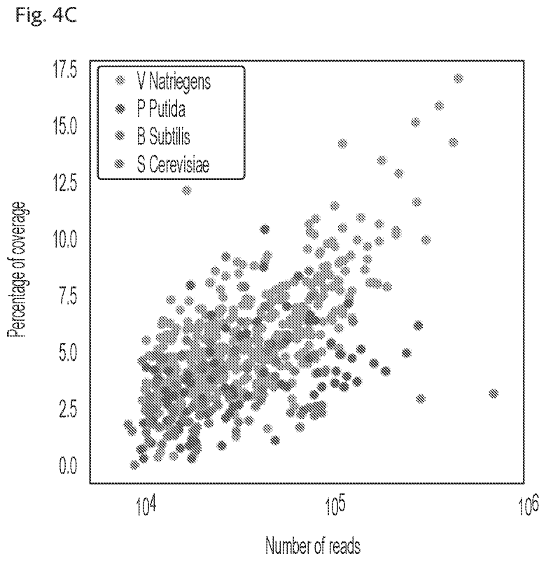

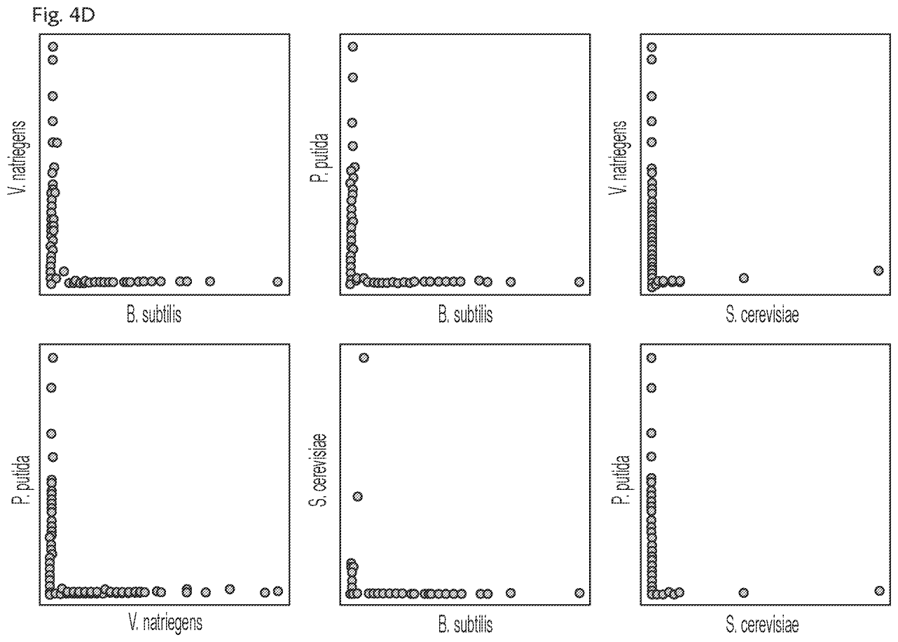

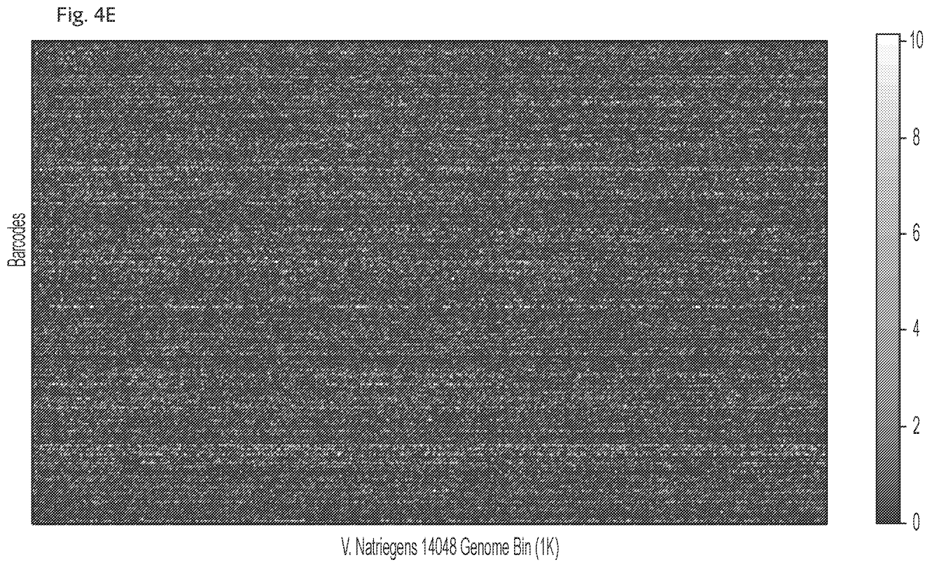

Using the methods provided herein, FIG. 4 shows the single cell genomic analysis of a sample consist of 4 microbiology (V. natriegens, P putida, B. subtilis, S. cerevisiae). Read count per barcode group plot (A) indicate the total number of cells passing filter. Histogram of barcode purity (B). Scatter plot of the percentage coverage of each barcode group with 95% purity vs the read counts (C). Cross talk between species in the barcode groups with 95% purity (D). Heatmap of read count across binned genome of all V natriegens barcode group indicate uniform coverage (E).

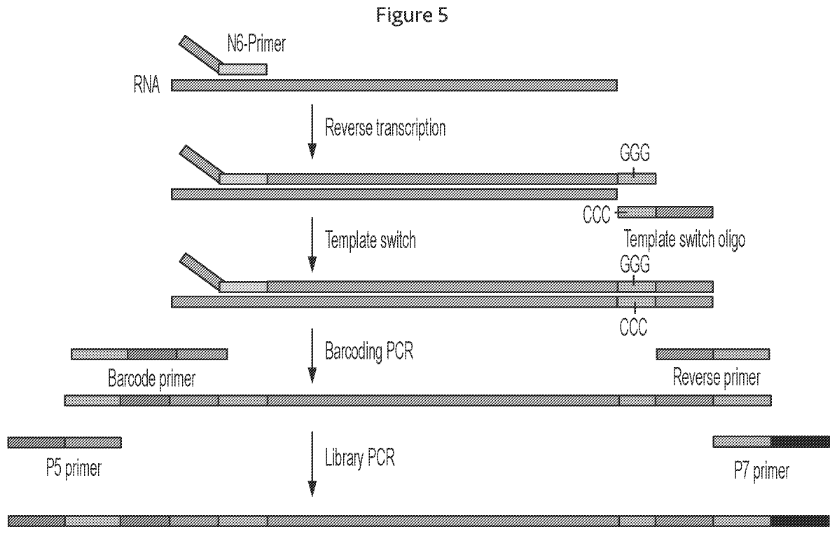

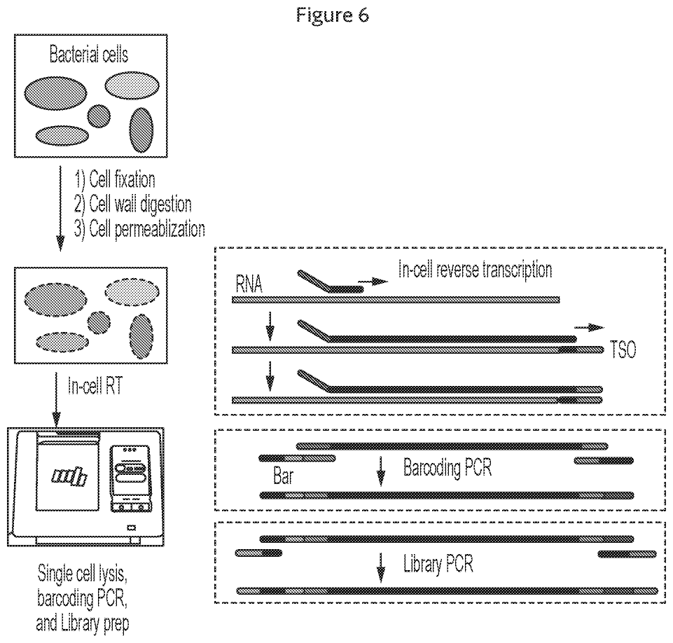

Additional embodiments are provided in FIGS. 5 and 6, as described herein.

### Example 3

### High-Throughput Single Microbe Whole Genome Sequencing on Commercially Available Microfluidics Platform

Microbiomes are microorganism communities within certain habitats, such as the human body. They have profound influences on almost all aspects of human life, such as health¹, medicine², food³, energy⁴ and environments⁵, thus are critical to study. As the next-generation sequencing cost continues to decline⁶, DNA sequencing-based microbiome analysis methods become more prevalent than the traditionally used cultivation and isolation-based methods₇. Because of the highly diverse and dynamic nature of microbiomes, population-based analysis methods such as marker gene sequencing and shotgun metagenomic sequencing are commonly used⁸. Marker gene sequencing, such as 16S and Internal Transcribed spacer (ITS) rRNA amplicon sequencing, take advantage of the conserved and variable regions of the marker genes for phylogenetic classification^{9,10}. Universal PCR primers are designed to target the conserved regions while the variable regions allow discrimination different species. Although convenient and cost efficient, marker gene sequencing has limitations such as limited resolution, low sensitivity, bias introduced by PCR amplification, and lack of functional gene information. To overcome the limitation, metagenomic techniques which sample the total DNA from a microorganism community is used to sequence an entire gene content¹¹. Compared with marker gene sequencing, metagenomics allows discovery of novel species and strains, comprehensive genome analysis and annotation, improved taxonomic and functional profiling, and genome assembly¹². However, due to the high diversity of the microbiome samples, the analysis of metagenomics also faces significant difficulties that require sophisticated computational and statistical tools¹³. For example, sequencing errors, uneven sequencing coverage, and presence of interspecies and intraspecies repetitive sequences and high-homology regions make genome assembly very challenging¹⁴. Also, assembled contigs binning, which refers to clustering the contigs into individual species or operational taxonomic units, remains as one of the most challenging tasks in metagenomics data analysis¹⁵.

Alternatively, single-cell genomics that can isolate and lyse individual microbes and subsequently amplify the DNA for sequencing is a better approach to overcome the limitation of marker gene and metagenomic sequencing. Single cell isolation using optical tweezers^{16,17}, flow cytometry^{18,19}, hydrogel matrices²⁰⁻²², microfluidics^{23,24}, or their combinations^{25,26} can process up to a few hundreds of single cells for sequencing. But this only represents limited members of microbiomes. A high-throughput single bacteria RNA sequencing method, microSPLIT²⁷, allowed microbe transcriptome analysis of thousands of individual cells. The microbe cells are fixed and permeabilized, and the RNA molecules are reverse transcribed into cDNA in situ. The cells are then processed by split-and-pool barcoding to generate single cell RNA reads. Unique expression states were observed that were not possible using population-based methods. However, due to the heterogeneous cell wall structures, which might need diverse fixation and permeabilization conditions, the methods are not easily transferable to microbiome samples and have only been applied to model species. Two high throughput single cell genomic DNA sequencing methods that are suitable for highly diverse communities, SiC-seq²⁸ and Microbe-seq²⁹, process tens of thousands of individual cells in parallel. Both methods take advantage of microfluidic droplet compartmentalization to process individual cells in a multi-step fashion to generate barcoded libraries. Sequencing of those libraries forms barcoded reads that can cluster into individual cells. SiC-seq allows in-silico flow cytometry based on characteristic sequences and gene distributions of sea water microbiomes. Microbe-seq can trace sub-strain dynamics, horizontal gene transfer, and bacteria-phage interactions within longitudinal collection of human fecal microbiomes. Those applications represent the revolutionary potential of high-throughput single microbe sequencing in the microbiome study. However, the complicated microfluidics operation requires extensive training and limits the accessibility to non-expert users.

The present Example evaluates several commercially available single cell sequencing platforms, and demonstrates a strategy, EASI-seq (Easily Accessible Single mIcrobe sequencing), for sequencing single microbes' genomic DNA by adopting, as one exemplary embodiment, Mission Bio's Tapestri microfluidic system. A non-microfluidic method was used to encapsulate single microbes in hydrogel beads. The cells were enzymatically lysed in hydrogel beads and the genomic DNA trapped in hydrogel beads were tagmented and barcoded in droplets. This method is validated with standard microbe synthetic communities and is applied to human fecal and environmental samples. The entire process requires no custom-build microfluidic devices and can be achieved in any biology laboratory with access to Mission Bio Tapestri. A bioinformatic pipeline for single cells clustering was also developed based on K-mer taxonomical detection. This unique single cell sequencing method can be easily adopted by microbiologists and allows high-throughput single microbe sequencing on a regular basis.

### A. High Throughput Single Microbe Genome Sequencing Workflow Design Based on Mission Bio Tapestri

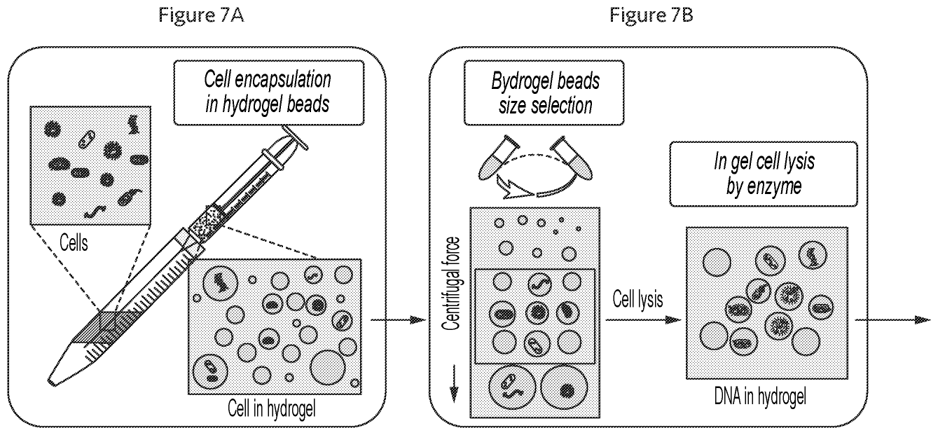

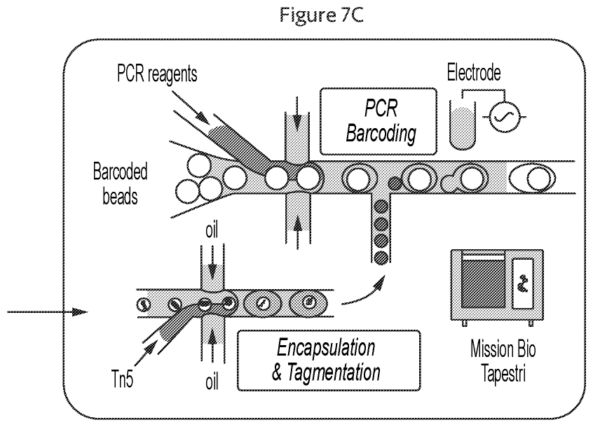

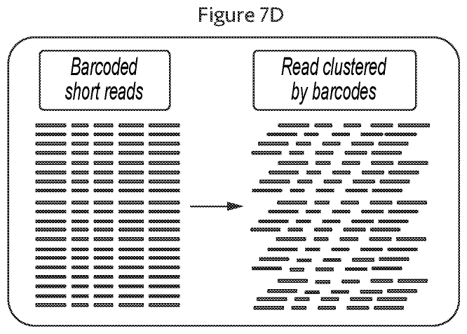

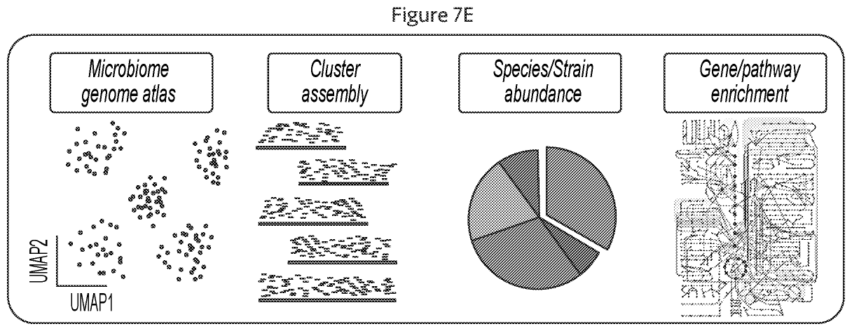

The present Example provides a workflow that allows a microbiologist to sequence thousands of single microorganisms in parallel on commercial platforms. The three main challenges are as follows. First, the presence of the bacteria walls requires harsh lysis conditions³⁰, and the genomic DNA must be purified to remove the inhibition effects of the cellular matter and the lysis buffer to the molecular biology reactions. Second, the long and circular genomic DNA need to be fragmented and universal adaptors should be introduced for amplification. Third, a proper barcoding strategy is required. To overcome those challenges, a multiple-step droplet workflow is necessary. For example, SiC-seq used 3 different microfluidic devices²⁸, while Microbe-seq needed five devices²⁹. Several droplet microfluidic single cell sequencing platforms were evaluated including Chromium Controller from 10× genomics, Tapestri from Mission Bio, Nadia from Dolomite Bio, InDrop system from 1CellBio, and ddSEQ from Bio-Rad. Tapestri was chosen in the present Example because it is the only system that allows 2-step partitioning (The first droplet step lysis the cells, while the second step pairs each droplet with a barcoding bead and PCR reagents.). After several iterations of protocol optimizations based on this 2-step system, the workflow of the present example is shown in FIG. 7 and represents one embodiment of the present disclosure. The overall design is encapsulating individual cells in hydrogel beads with a microfluidics free method and lysing the cell within the hydrogel beads. The genomic DNA purified in each hydrogel bead are then tagmented and barcoded for sequencing on Tapestri.

We first isolate the cells from microbiome samples and resuspend them in acrylamide solution. Then we generate heterogeneous emulsions with cells by passing the cell suspension and oil through a syringe needle (FIG. 7A). After gelation, we remove the oil to collect the hydrogel beads with individual cells. In contrast to SiC-seq, where a microfluidic device is used to generate agarose hydrogel beads for cell encapsulation²⁸, we use a microfluidic free method to generate the hydrogel beads to make it more accessible to non-microfluidic experts. Because the heterogeneous hydrogel bead sizes ranges from more than 100 um to smaller than 1 um, where the large beads might contain multiple cells and DNA could leak out from the small sized beads, the hydrogels were size-selected with differential centrifugation to 20 to 5 μm (FIG. 7B). Those size ranges are similar to the sizes of mammalian cells, which can be directly processed on typical commercial single cell platforms designed for mammalian cells, including Tapestri. Polyacrylamide was used as the gel matrix and N,N′-bis(acryloyl)cystamine as cleavable crosslinker, instead of hydrogen bond-based hydrogel, such as agarose^{25,26,28}, because the cells escape from agarose gel beads during the lysis treatment, especially from the smaller ones. The cells were lysed by sequentially soaking the hydrogel beads in cell wall digestion enzymes and protein lytic enzymes. The small pore-size of hydrogel allows proteins and other molecules freely diffuse except long DNA molecules, after washing only genomic DNA retained in the hydrogel beads (FIG. 7B).

Genomic DNA molecules within the hydrogels are processed using the Mission Bio Tapestri's two-stage microfluidic system or lab-made microfluidic chips with the same functions (FIG. 7C). The first droplet step introduces universal adaptors for amplification by tagmentation, while the second step pairs each droplet with a barcoding bead and PCR reagents. In the first droplet step, DTT is added in the tagmentation buffer to break the disulfide bond in the crosslinker to allow complete release of the genomic DNA. The Tn5 transposase is assembled with both forward adaptors that match the Mission Bio barcoding primer 3′ constant region. The genomic DNA is fragmented, and the adaptors are introduced to each fragment. The droplets are then merged with barcode beads and PCR reagents. The barcode primers are released from the barcode beads upon UV treatment and are linked to the DNA fragments after thermal cycling.

The barcoded amplicons are further amplified using Illumina index sequencing adapter to form a sequencing library. Sequencing the library produces reads that can be clustered into single cell groups by unique barcodes (FIG. 7D). We demonstrate the method with high-throughput microbiome genome atlas analysis, as well as cluster-based genome assembly, strain identification, and pathway analysis (FIG. 7E)

B. Single Cell Accuracy and Sensitivity Validation with Microbial Communities

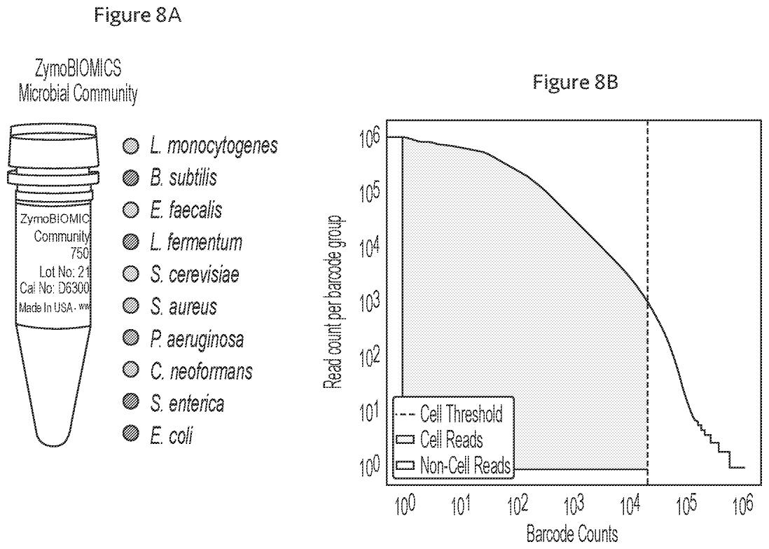

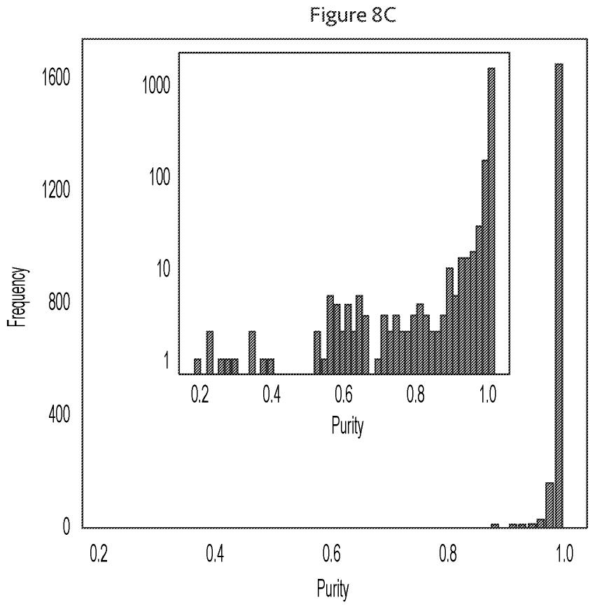

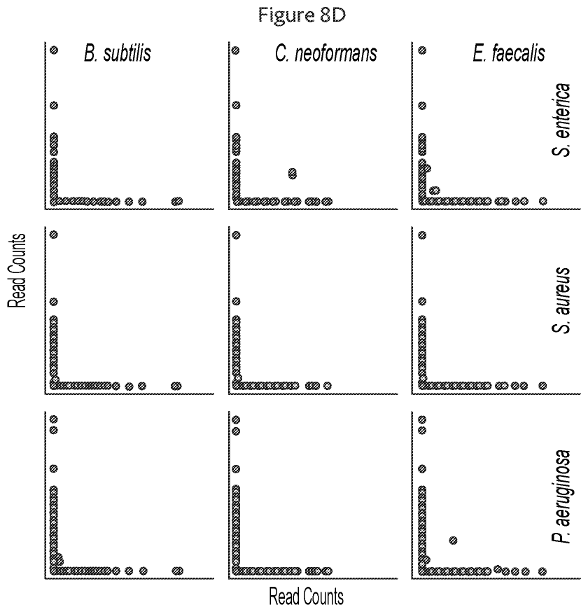

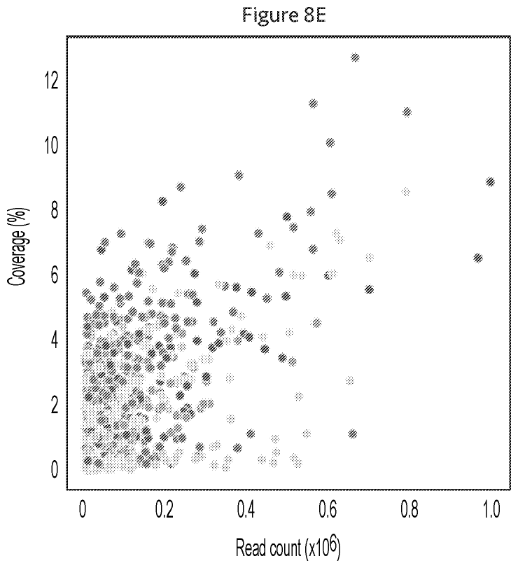

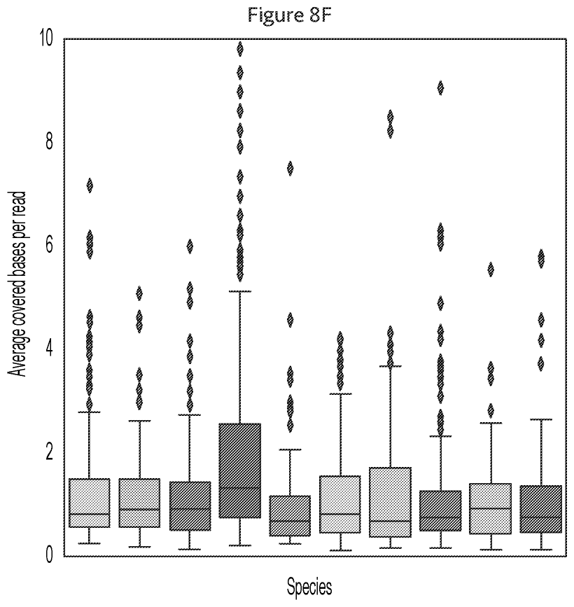

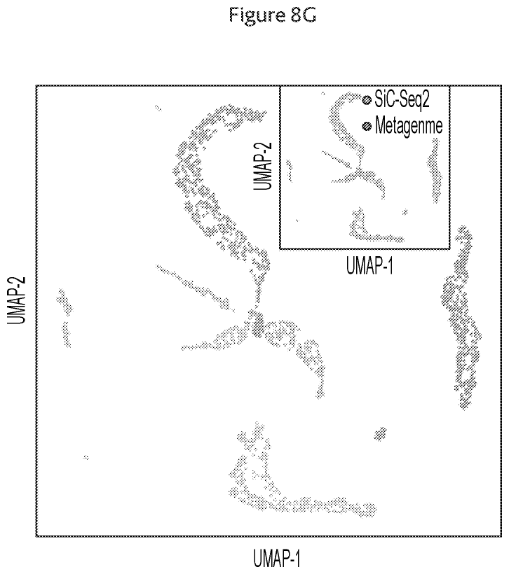

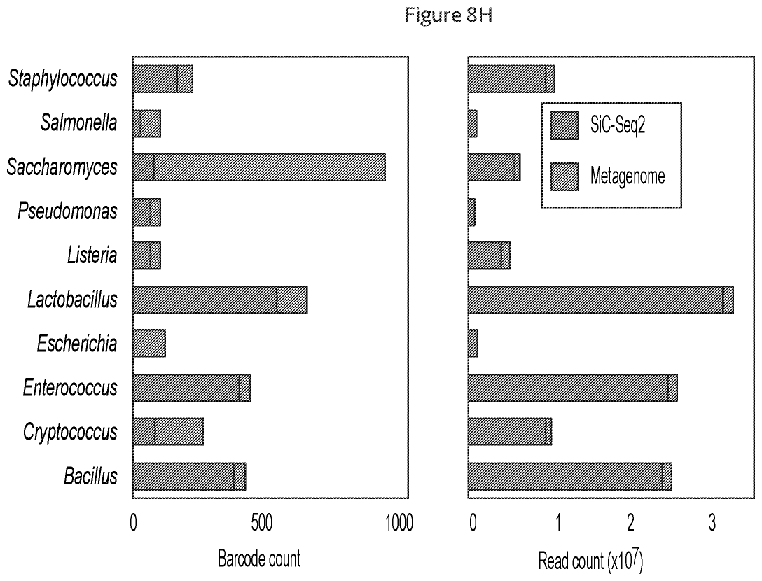

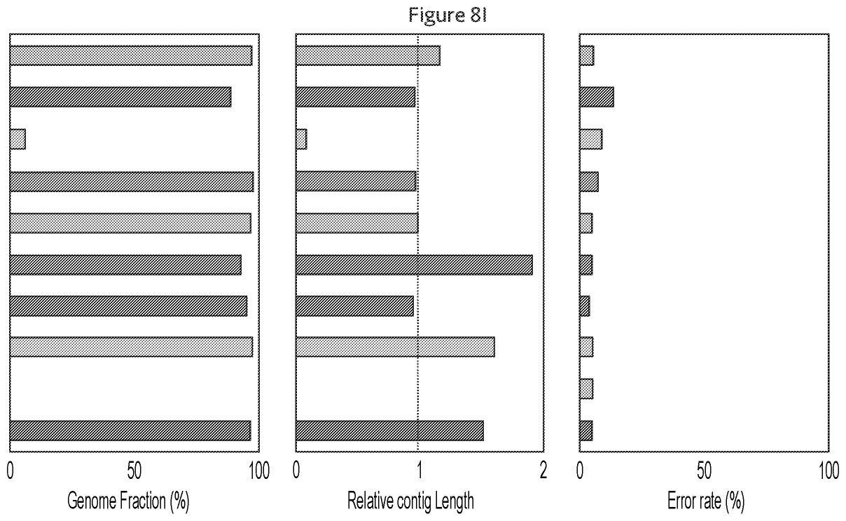

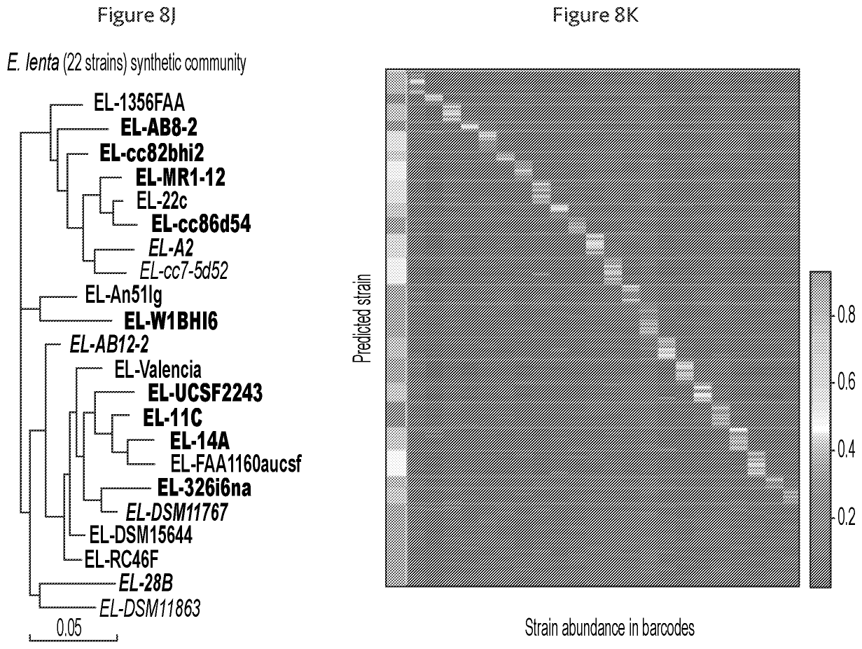

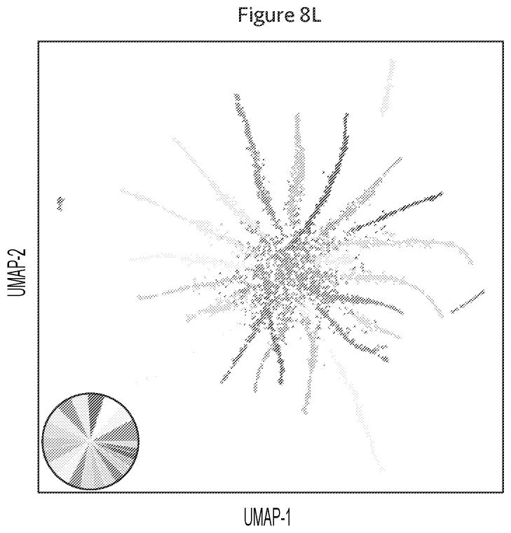

To validate that the EASI-seq generates single cell barcode groups, a synthetic microbial community was used (Zymobiomic standard community), which consists of 3 Gram-negative bacteria, 5 Gram-Positive bacteria and 2 yeasts (FIG. 8A). The community was processed using EASI-seq and sequence the library on Illumina platform. 238,362,515 pair-end reads of 150 bp were generated after quality filtering and group the read reads into individual files by barcode sequences. To ensure the quality of each barcode group, the barcode groups were filtered according to read counts (FIG. 8B) and mapping rates to the reference genomes of 10 species. 1835 of barcode groups are recovered, each containing an average of 71,684 reads. The purity of each barcode group was measured, which is defined as the percent of reads that aligned to a dominant species. 86.16% of barcodes were found to have a purity of more than 90% (FIG. 8C) and most of the barcode groups dominantly align to only one species (FIG. 8D and FIG. S6 Zymo all barnyard plot). This suggests EASI-seq has true single cell accuracy. To assess the performance of EASI-seq, the coverage and unique based covers per reads of each barcode group was calculated. The average coverage is 0.4444% for bacteria species and 0.0313% for yeast species (FIG. 8E). Each barcode gains 1.14±0.28 unique covered bases per reads, and the similar bases covered per reads for different species indicate that EASI-seq is not biased against any species (FIG. 8F).

Because only a limited fraction of microbiome samples has a reference genome available, the possibility to annotate each barcode group without the need of a reference genome was further explored. Since each barcode only covers about 0.44% of the genome (FIG. 8E), it is likely that barcode groups of the same species could have completely different sets of reads. Also, because an amplification step before DNA fragmentation was not used, the reads from one barcode group do not overlap, thus are not able to be assembled. To overcome these issues and correctly assign each barcode to the right taxonomic group, Taxonomic Discovery Algorithm (TDA) is used in which each barcode group is treated as one metagenomic file and a taxonomical estimation tool is used to predict the abundance of each taxonomy. The taxonomic abundance profiles of different barcode groups belonging to the same species should be similar even though the barcode groups might contain completely different read sets. The barcode groups with similar taxonomic abundance profiles by TDA can be treated as the same type of cells. To find a taxonomic estimation tool that are fit for EASI-seq barcode group data structure, the 3 different types of taxonomical estimation tools were evaluated that provide relative abundance of OTUs using simulation data: K-mer based tools (Kraken2/Bracken^{31,32}), marker gene-based tool (MetaPhlAn3³³), and Protein based tool (Kaiju³⁴) using simulation data. The accuracy of barcode purity prediction and taxonomical annotation was calculated, as well as the barcode recovery rate with filtering. The k-mer based Kraken2 showed the best performance. (Detailed discussion provided in supplementary information). Kraken2 was chosen for the barcode evaluation in the TDA. To evaluate the TDA performance, a synthetic community was used for data analysis. Firstly, all barcode groups were profiled in the ZymoBiomics sample using Kraken2 with PlusPF database. Secondly, the ambiguity barcode groups with 1% or less mapping rate were removed and obtained 1805 barcode groups passing filter. Thirdly, Bracken was used to re-estimated the genus abundances of all the barcode groups. Lastly, the genus abundances of the filtered barcode groups are combined as a vector. To visualize the data structure, uniform manifold approximation and projection (UMAP) to reduce the dimension of the genus abundance vector was used. 10 clusters were identified that are mapped to the 10 species (FIG. 2g). We also compare TAD with reference genome-based identification and find 97.34% of barcode groups are correctly identified using TAD. This high accuracy suggests that the Taxonomic Discovery Algorithm is suitable for reference-independent barcode annotation.

For some of the clusters we identified, there are not enough reads to assemble and analyze the functions of the genome. For example, only 4 barcode groups are identified as P. aeruginosa, and the total read count is only 34,021. To overcome the limitation, the metagenome dataset of the same sample was integrated with the EASI-seq data. First, each contig assembled from the metagenomic data (FIG. S9a Zymo metagenomic assembly statistics and filtering) of the same sample as individual barcode group was treated and extracted the paired-end reads from each contig. The contig barcode groups were processed with TDA. 1427 out of 4844 contigs were identified that have more than 90% purity at genus level. Those “metagenomic barcodes” are overlayed perfectly with the EASI-seq barcodes (FIG. 8G inset). With the additional barcodes and reads (FIG. 8F), all the reads were assembled in each cluster to cover 94.31±4.92% of the bacterial species and 2.74±3.24% of the yeast species (FIG. 8I). The relative total contig lengths are close to 1 and the overall error rate is 5.87±2.97% for all species. Those results suggest the TDA is also suitable for integration of single cell data and metagenomic data sets.

Strain Identification with Microbial Communities

Strain identification is an important but challenging task in microbiome study. To evaluate the ability of EASI-seq to identify strains of the same species, a synthetic community consisting of equally mixed 22 strains of Eggerthella lenta (E. lenta)³⁵ with EASI-seq (FIG. 8J) was analyzed. The library was sequenced on Illumina platform and generated 105,896,184 pair-end reads of 150 bp after quality filtering. The reads were grouped into individual files by barcode sequences and aligned them to the 22 reference genomes. To ensure the read quality, the barcode groups were filtered based on read counts and mapping rate and recovered 5345 barcode groups with a total of 101,760,151 reads. To identify the correct stain for each barcode group, a method similar to differential transcripts expression estimation in RNA sequencing was used to estimate the abundance of the 22 strains in each barcode group³⁶. First, the reads in each barcode group were mapped to all the 22 reference genomes and all possible alignments are recorded. The probabilities to each alignment were calculated with the quality scores and mismatches for every alignment of a read and with the assumption of a Log-Normal read distribution (parse Alignment). The “expression level” of the 22 strains in each barcode group are then calculated by Variational Bayes algorithm (estimate VBExpression). A specific strain is assigned to a barcode group if the abundance of that strain is ≥15% (FIG. 8K). About 19% of the barcode groups (1020 out of 5345) are not identified. The reason could be that the unidentified barcode groups contain either more than one strain or have less reads covering the strain specific regions. The strains were visualized using UMAP clustering as well as a pair plot of the abundance estimation (FIG. 8L). The clear separation between different strains confirms EASI-seq's strain level resolution.

C. Human Fecal Microbiome Analysis with EASI-Seq

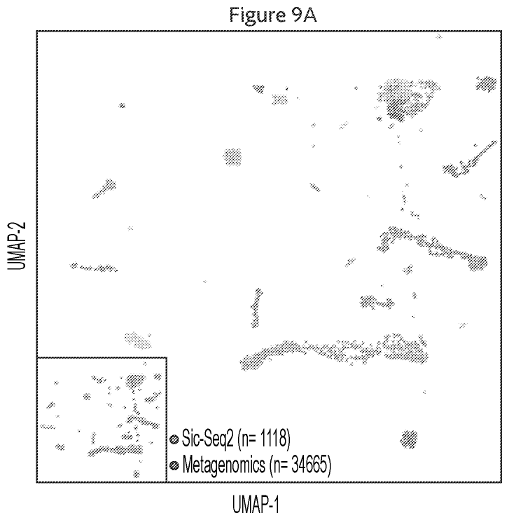

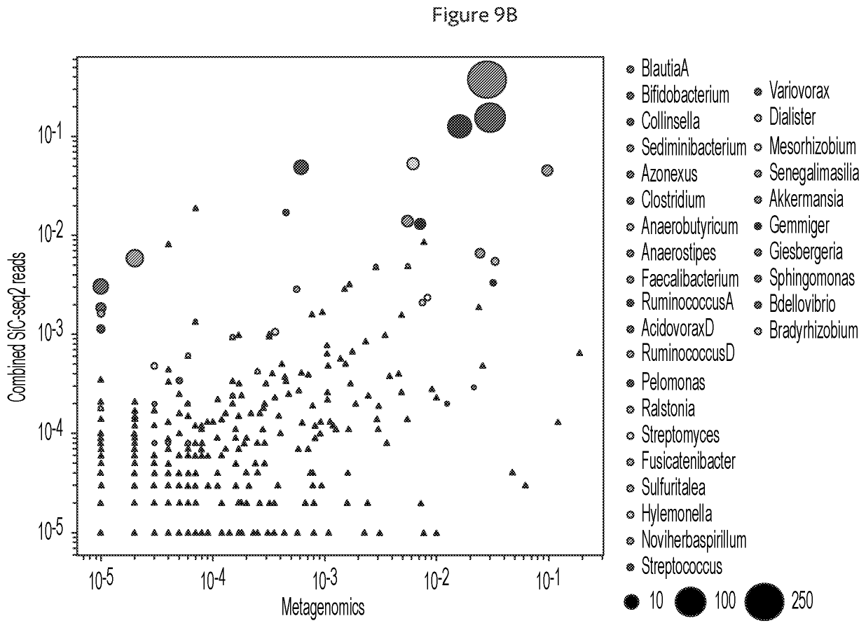

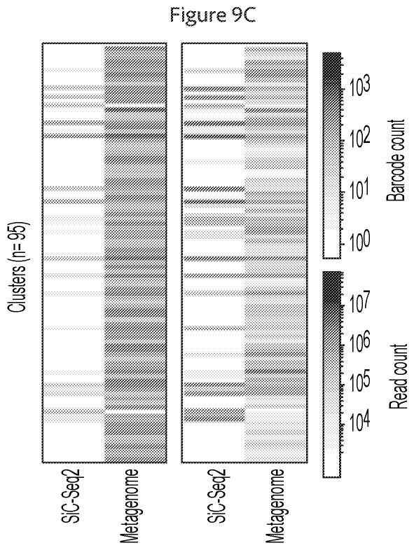

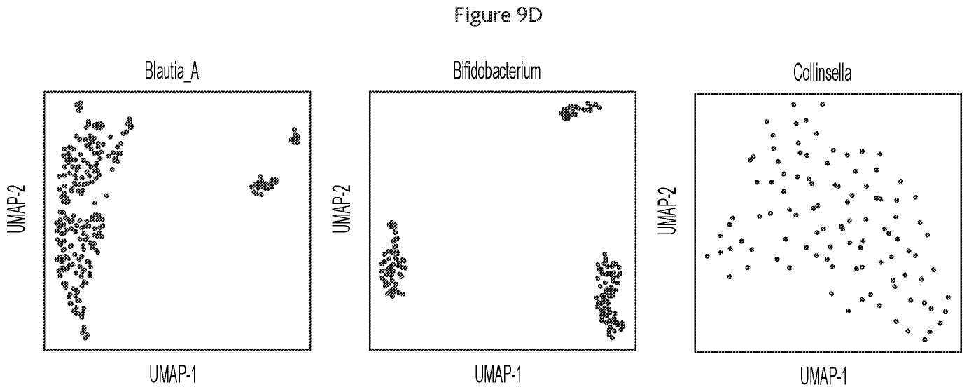

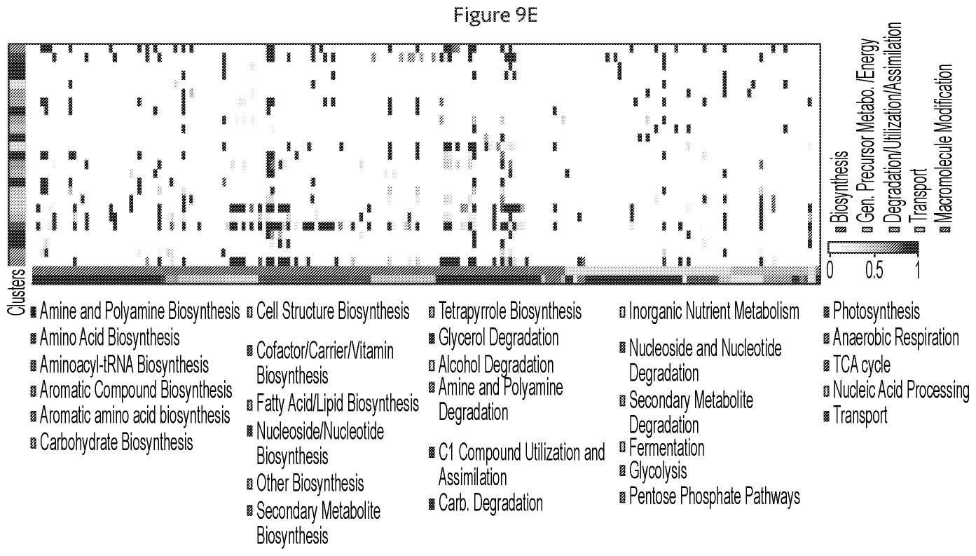

To explore the utility of EASI-seq, to the method was applied to the human microbiome samples. Microbial cells were isolated from a healthy donor's fecal sample using density centrifugation. A single cell sequencing library for the fecal microbiome cells was generated using EASI-seq. After quality filtering. 232,705,096 pair-end reads of 150 bp were recovered. The reads were grouped into single files by barcode sequences and filter the barcode groups according to read counts (>1000 reads) and genus level purity estimated by Kraken2 (>80%). The recovered 1118 barcode groups contain an average of 153,660 reads. To increase the overall coverage, the EASI-seq data was integrated with metagenomic data of the same sample. The short reads associated from the 34,665 contigs assembled from the same sample's metagenomic reads were extracted and treated them as individual barcode groups. The contig barcode groups were filtered based on classified read percentage by Kraken2³¹ and the genus level purity before integrating them with the EASI-seq barcodes. From the combined barcode groups, 95 clusters were identified at genus level (Fig. (A) and the detailed barcode counts and read count is plotted as heatmaps in FIG. 9C.

To analyze the microbial diversity in the sample, we compare the relative genus abundances of aggregated EASI-seq reads with that of metagenomic shotgun sequencing (FIG. 9A). several genera's abundances in the EASI-seq reads are significantly reduced, including Bacteroides, Phocaeicola, Parabacteroides, Akkermansia, and Alistipes. This is likely caused by cell isolation process. During the centrifugation-based cell isolation process³⁷, a 5 μm size filter was used to remove large particles. The cells with properties such as sticky to fecal particles or tend to form big aggregates might be removed from the sample. This is supported by the observation that certain species of Bacteroides can adhere to partially digested food particles³⁸. Another reason for this bias could be introduced by the sample storage at −80° C., which is known to increase the composition of Blautia and Collinsella while decrease that of Bacteroides and Alistipes³⁹. In one embodiment, the relative abundance accuracy is improved by using fresh collected samples.

To address different species within the genus level clusters, the genus-level clusters were divided into subclusters by UMAP using species abundance from TDA (FIG. 9D). For example, the three clusters with the most barcode groups: Blautia-A, Bifidobacterium and Collinsella can be categorized into 10, 7, and 6 sub-clusters, respectively, this suggests there are possible 10, 7, and 6 species in the corresponding genus present in this sample.

To understand the genome function of each cluster in the human microbiome, the genome pathway of each cluster using was evaluated Humann2⁴⁰ with the MetaCyc pathway database⁴¹. We found that different clusters possess distinct pathways (FIG. 9E). For example, Blautia_A and Bifidobacterium have different sets of pathways for carbohydrate degradation and fatty acid/lipid biosynthesis, which suggest their nutrient source and role in fatty acid production are different in the human guts.

### D. Environmental Microbiome Analysis Using EASI-Seq

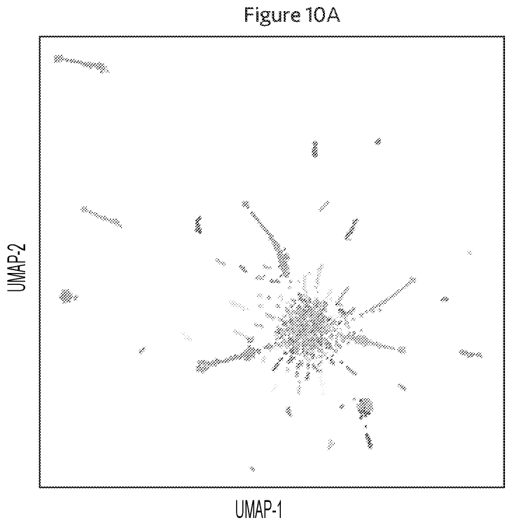

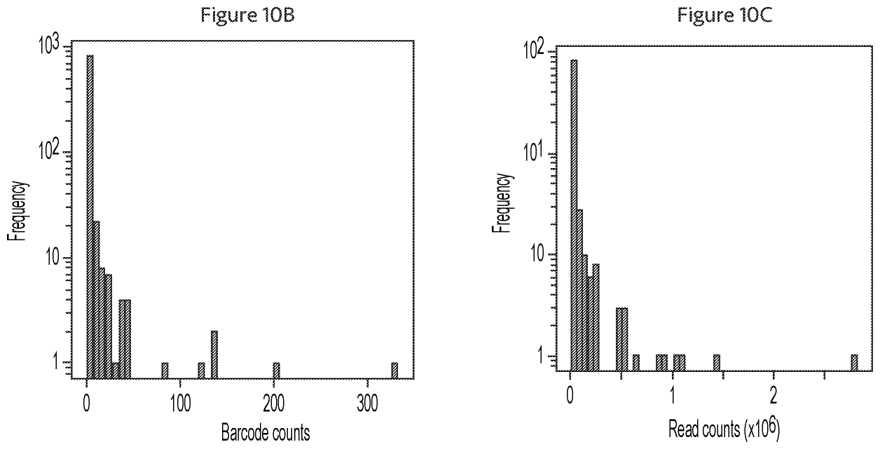

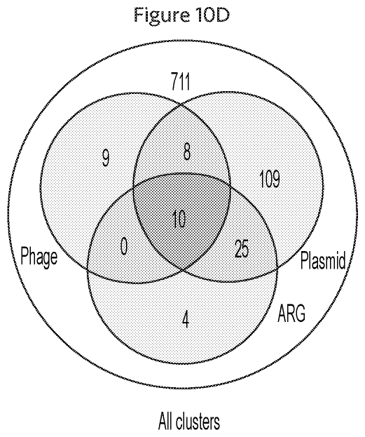

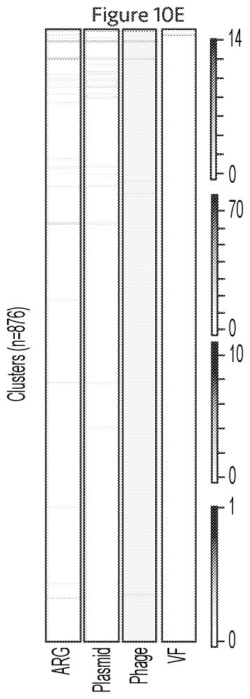

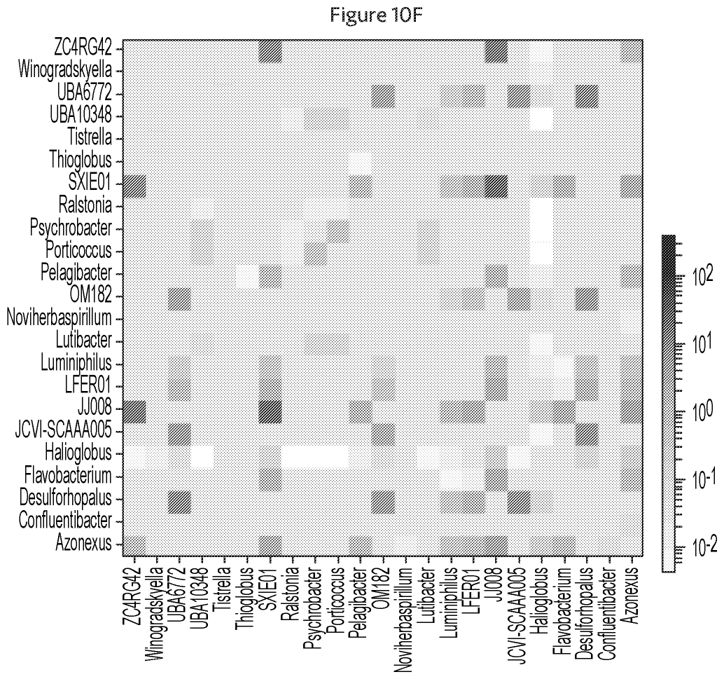

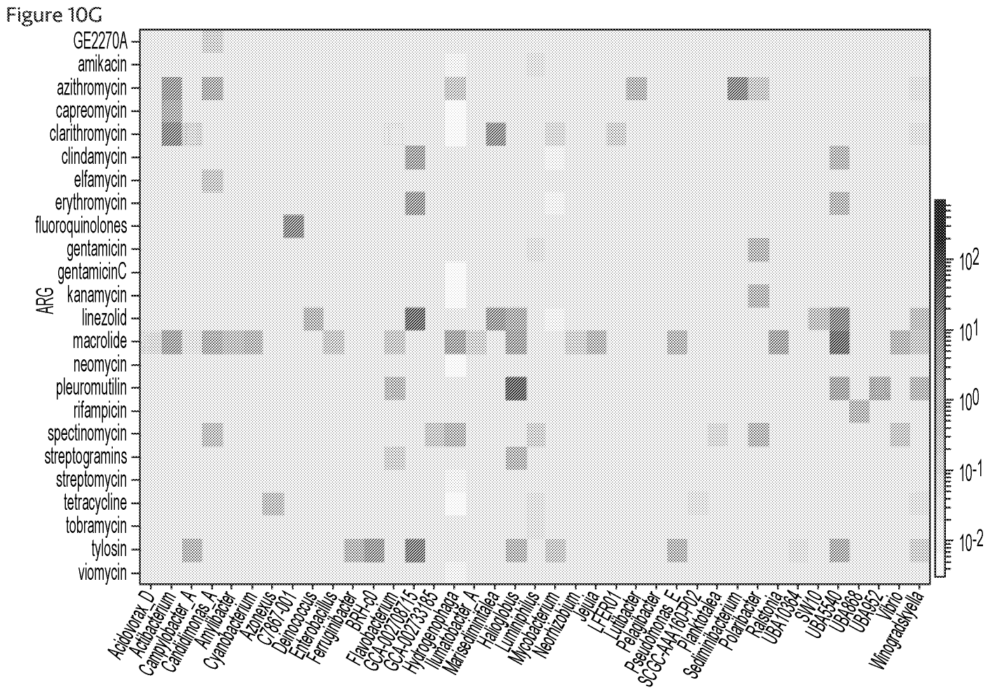

The robustness of EASI-seq was further tested with environmental samples. Coastal seawater near was collected San Francisco and cells were isolated using filtration. Cells were processed with EASI-seq and sequence the library on Illumina platform, yielding 34,090,184 pair-end reads of 150 bp after quality filtering. The reads are grouped into individual files by barcode sequences. To ensure high quality, the barcode groups were filtered according to mapping rates to the K-mer database in Kraken2 and recovered 3235 barcode groups with an average of 8153 reads. Using TDA, we discover 876 genus level clusters (FIG. 10A), of which 96.77% are bacteria, and 3.23% are archaea.

The barcoded reads of EASI-seq allow direct association between cellular genome DNA and extrachromosomal DNA or mobile elements, such as plasmid, phage genes, antibiotic resist genes, or virulence factors, while is still challenging to study using metagenomics. The extrachromosomal DNA and mobile elements containing barcodes was searched in the environmental sample and identified 388 plasmid, 28 phages, 42 antibiotic resistant genes, and 1 virulence factor in 165 barcode groups (FIG. 10D-E). There are 42 known antibiotic resistant gene are found in 39 clusters. The antibiotic resistance gene distribution among taxa in our data is shown in FIG. 10G. For example, Halioglobus cluster carries the genes with resistance to 12 different antibiotics. The result is partially confirmed by a study⁴² that one strain of Halioglobus sediminis has shown resistance to 5 antibiotics, including 2 are also found by EASI-seq. The present method thus provides a new way for antibiotic resistance gene screening.

Horizontal gene transfer is often mediated by cross-infection of bacteriophages⁴³. To analyze the relative potential for transduction among taxa, 28 phage sequence in 27 clusters were identified. The likelihood of the transduction between two taxa is proportional to the probability of the two taxa share the same phage infection. The interaction between different taxa was plotted as a heatmap (FIG. 10F). Two genera were found: JJ008 (from Family Chitinophagaceae) and SXIE01 (from Family PHBI01) has the highest transduction potential.

### E. Discussion

For the past decade, single cell sequencing has completely transformed the mammalian biology study. Since the first single cell transcriptome study by in 2009 Tang et al.⁴⁴, several commercial single cell platforms became available and now single cell RNA-seq became a routine and standard lab operation. With single cell RNA seq, research can assess individual cell expression within heterogeneous population, identify unique regulation between genes, and track cell lineage trajectories and many other analyses that are not possible with population-based bulk measurements⁴⁵. Later, the technological improvement enabled single cell multiomics analysis, which means measurement of multiple modalities in single cells, such as RNA plus protein⁴⁶, or genomic DNA plus protein⁴⁷. Those approaches provided unprecedented opportunities to a systemic understand the fundamental cell biology. More recently developed spatially resolved transcriptomics have enabled the connection of gene expression to spatial organization of individual cells within tissues⁴⁸. Of course, as the expansion of the single cell sequencing techniques and rapid growth of data generation, the bioinformatic toolkits have also been developing. Those pipelines address critical challenges in single cell analysis, including data preprocessing, alignment, quality check, normalization, dimension reduction, differential expression, pseudo-time construction, RNA velocity, and batch integration⁴⁹. However, for microbiomes, the development of single cell sequencing techniques has been far lagged. Although there are great scientific values for single microbe sequencing, there are only a few reported methods and none of those are commercially available due to various technical challenges.

Here, a non-microfluidic method was used to encapsulate single cell in hydrogel beads that allows lysis and DNA purification. By adopting the 2-step microfluidic platform from Mission Bio, the genomic DNA of individual microbes was barcoded. A bioinformatic pipeline was developed to aggregate barcode groups at different taxa levels. With EASI-seq, the intraspecies variability was analyzed and closely related strains were identified, directly measured the gene distribution across different taxa, grouped the plasmids with the genomic DNA. Those tasks are still difficult to achieve by metagenomics. The application of EASI-seq thus allows microbiome cell atlasing, improving the therapeutic microbiome development, and enables microbiome ecosystem analysis at improved resolution. The single cell reads were integrated with metagenomic reads to improve the genome assembly quality. The present methods did not show any bias between bacteria and yeast, and archaea were also barcoded in one sample. EASI-seq can thus be extended to other single celled species, such as protozoa, unicellular algae, or even isolated cells from multicellular species.

### F. Methods

### 1. Microbiome Samples Processing

a. Synthetic Community

ZymoBIOMICS standard (Zymo, D6300) was stored at −80° C. until use. 100 μL of ZymoBIOMICS was washed with 4 mL of PBS for 3 times to remove the storage buffer. The cell density are measured with Countess™ cell counting slides (Thermo Fisher, C10228) using EVOS microscope. The cells were resuspended to 100 million per mL in PBS.

22 E. lenta strains (were cultured in appropriate media (10.1016/j.chom.2020.04.006) and equally mixed by CFU counting in culture media. The mixed cells were stored at −80° C. until use. The cells were washed 3 times to remove the storage media, and filtered with 5 μm syringe filter to remove cell aggregates. After cell counting, the cells were resuspended to 100 million per mL in PBS.

b. Human Microbiome and Cell Isolation

Fecal sample from health donor is stored at −80° C. until use. Cell isolation was performed according to previously reported protocol (hevia 2015). About 0.5 g of fecal sample was homogenized in PBS (10 mL). The suspension is filtered through a 50 μm cell strainer (Corning, 431752) to remove the large fecal particles and loaded into a 15 mL centrifuge tube with 3.5 mL of 80% Nycodenz® solution (Cosmo Bio USA, AXS-1002424). After centrifuge at 4700×g for 40 min at 4° C., the layer corresponding to cells was collected by pipetting. The cells were washed with PBS for 3 times, filtered with 5 μm syringe filter, and then resuspended to 100 million per mL in PBS.

c. Ocean Water Microbiome and Cell Isolation

Sea water was collected at Pacific coastline near San Francisco (GPS coordinate: 37.7354373 N, 122.5081862 W) by submerging a 1000 mL sterile bottle into the ocean. The sea water was transferred to the lab on ice. The cell was isolated according to the published protocol (SiC-Seq). Briefly, the sea water was first filtered through a 50 μm cell strainer (Corning, 431752) to remove sands or other large particles. The suspension was then filtered by a 0.45 μm vacuum filter (Millipore, SCHVU01RE) to capture the cells on the membrane. The membrane was cut off from the filter with a sterile razor blade and transferred a 15 mL centrifuge tube with 5 mL PBS. The cells were released from the membrane by vortexing the tube at maximum speed for 2 min. The cells were washed with 10 mL PBS for 3 times and passed through a 5 μm syringe filter to remove remaining virus or large particles. The cells were resuspended to 100 million per mL in PBS.

### 2. Microfluidics Device Fabrication

Microfluidics devices were fabricated with standard photolithography and soft lithography method. Custom device fabrication is not necessary for the single cell sequencing using Mission Bio Tapestri but used for workflow optimization. Master photomask was designed using AutoCAD and printed at 12,000 DPI (CAD/Art Services, Bandon, OR). To make the master structure, SU8 Photoresist (MicroChem, SU8 3025 and SU8 3050) were spin coated on three-inch silicon wafers (University Wafer), soft baking at 95° C. for 10 to 20 min, UV-treated through the photomasks for 3 min, hard baked at 95° C. for 5 to 10 min and developed in propylene glycol monomethyl ether acetate (Sigma Aldrich). For the microfluidic devices, poly(dimethylsiloxane) (PDMS) (Dow Corning, Sylgard 184) and curing agent were mixed in 10:1 ratio, degassed and poured over the master structure, baked at 65° C. for 4 h to cure, and peeled off from the wafer. After hole punched with a 0.75 mm biopsy puncher, the devices were plasma treated and bonded to glass slides. The channels were treated with Aquapel (PPG industry) to for hydrophobic surface and dried by baking at 65° C. for 10 min.

### 3. Single Cell Genomic DNA Isolation in Hydrogel Beads

a. Cell Encapsulation in Hydrogel Beads

500 μL cell suspension (100 million per mL in PBS) was mixed with 500 μL hydrogel precursor solution (12% acrylamide, 1% BAC, 20 mM Tris, 0.6% sodium persulfate, and 20 mM NaCl in H₂O) in a 15 mL centrifuge tube. 1 mL HFE 7500 with 2% surfactant (008-FluoroSurfactant, RanBiotechnologies) was added to the cell/hydrogel precursor mixture. Emulsion was formed by passing the oil/aqueous mixture 5 times through the needle. 20 μL of TMEDA (tetramethylethylenediamine, Sigma) was added into the emulsion and the emulsion was incubated at 70° C. for 30 min and at room temperature for overnight for gelation. The emulsion can be stored at 4° C. for up to 1 week.

The emulsion was centrifuged at 1000 RCF for 1 min and the bottom oil layer was removed by using a gel loading tip. 1 mL of 20% PFO (1H,1H,2H,2H-perfluoro-1-octanol, Sigma, 370533) and 5 mL of PBST buffer (0.4% tween 20 in PBS) were added into the emulsion. The mixture was vortexed at maximum speed for 1 min break the emulsion and centrifuged at 1000 RCF for 5 min. Any remaining oil was removed by pipetting through a gel-loading tip.

b. Hydrogel Size Selection

Differential velocity centrifugation was performed to select the hydrogel beads from previous step within the diameter between 5 to 15 μm. The hydrogel beads were resuspended in 14 mL high density buffer (40% sucrose in PBS with 0.4% tween 20). First, the beads were centrifuged at 1000 RCF for 5 min to pellet large gels. The supernatant was transferred to a new 15 mL tube and centrifuged at 3000 RCF for 10 min to pellet the right sized beads. The supernatant (still containing beads smaller than 5 μm) was discarded and the pelleted beads were washed 3 times with PBST to remove the high-density buffer.

c. Cell Lysis in Hydrogel Beads

100 μL of size selected beads were treated in 1 mL cell wall digestion buffer (TE buffer solution containing 2.5 mM EDTA, 10 mM NaCl, 2 U zymolyase, 5 U Lysostaphin, 50 U mutanolysin, and 20 mg Lysozyme) at 37° C. overnight. The beads were then pelleted by centrifugated at 3000 RCF for 10 min and washed with PBST for 3 times. The beads were then treated in 1 mL protein digestion solution (TE buffer with 4 U of Proteinase K, 1% triton X100 and 100 mM of NaCl) at 50° C. for 30 min. Following lysis, the beads were thoroughly washed with PBST, 100% EtOH, and PBST 3 times to ensure complete removal of proteinase K and other chemicals which may inhibit the downstream reactions. The beads were then filtered with 10 μm cell strainer and ready for droplet tagmentation.

### 4. Single Cell Tagmentation and Barcoding in Droplet Microfluidics

Microfluidic droplet encapsulation, tagmentation, and barcoding PCR were performed on commercial single-cell DNA genotyping platform (Mission Bio, Tapestri) or custom build microfluidic devices with the same functions.

a. Tagmentation Reagents

25 μL Tn5-Fwd-oligo GTA CTC GCA GTA GTC AGA TGT GTA TAA GAG ACA G (SEQ ID NO: 1)(100 nM, IDT), 25 μL, Tn5-Rev-oligo TAC CCT TCC AAT TTA ACC CTC CAA GAT GTG TAT AAG AGA CAG (SEQ ID NO: 2) (100 nM, IDT), and 25 μL Blocked ME Complement/5Phos/C*T* G*T*C* T*C*T* T*A*T* A*C*A*/3ddC/(SEQ ID NO: 3)(200 nM, IDT) and 25 μL Tris buffer were mixed well in a PCR tube by pipetting. The mixture was incubated on a PCR thermal cycler with the following program: 85° C. for 2 min, cools to 20° C. with a ramping rate at 0.1° C./s, 20° C. for 1 min, then hold at 4° C. with lid at 105° C. 100 uL of glycerol was added into the annealed oligo. Unloaded Tn5 protein (1 mg/mL, expressed by QB3 MacroLab, Berkeley, CA), dilution buffer (50% Glycerol, 100 mM NaCl, 0.1 mM EDTA, 1 mM DTT, and 0.1% NP40 in 50 mM Tris-HCl pH 7.5 buffer), and the pre-annealed adapter/glycerol mix were mixed at 1:1:2 ratio by pipetting. The mixture was incubated at room temperature for 30 min then stored at −20° C. until use. For droplet tagmentation, equal amount of assembled Tn5 and tagmentation buffer (10 mM MgCl2, 10 mM DTT in 20 mM TAPS pH 7.0 buffer) were mixed.

b. Droplet Tagmentation

In the first droplet step, the tagmentation reagents (0.125 mg/mL assembled Tn5, 10 mM MgCl2, and 10 mM DTT in 20 mM TAPS pH 7.0 buffer) and the genomic DNA in hydrogel beads (equivalent to 3 million cells per mL) in 10 mM MgCl₂, 1% NP40, 17% Optiprep, and 20 mM TAPS pH 7.0 buffer were co-flowed in the microfluidic devices to form droplets.

In case of using Tapestri, the MissionBio Tapestri DNA cartridge and a 0.2 mL PCR tube were mounted onto the Tapestri instrument. 50 μL beads solution, 50 μL tagmentation reagents, and 200 μL encapsulation oil were load in the cell well (reservoir 1), lysis buffer well (reservoir 2), and encapsulation well (reservoir 3) on the Tapestri DNA cartridge, respectively. The Encapsulation program was used for droplet generation. The droplets were collected into a PCR tube.

For custom build microfluidic device, the beads solution, the tagmentation reagents, and 5% (w/w) PEG-PFPE surfactant (Ran Biotechnologies) in HFE 7500(3M) were loaded into three syringes and placed on syringe pumps. The syringes were connected to the co-flow droplet generator device via PTFE tubing. The pumps were controlled by a Python script (https://github.com/AbateLab/Pump-Control-Program) to pump bead solution at 200 μL/h, tagmentation reagents at 200 μL/h and oil at 600 μL/h to generate droplets. The droplets were collected into PCR tubes.

The droplets generated by either method are incubated at 37° C. for 1 h, 50° C. for 1 h, and hold at 4° C. to ensure hydrogel melting and Tn5 complete reacting.

c. Droplet Barcoding PCR

The tagmentation droplets from the previous were merged with PCR reagents and barcode beads for barcoding with either Tapestri or custom build microfluidic devices.

In case of using Tapestri, 8 PCR tubes and DNA cartridge were mounted onto the Tapestri instrument. Electrode solutions were loaded into electrode wells (200 μL and 500 μL in reservoirs 4 and 5, respectively). After running the Priming program, 5 μL of reverse primer was mixed with 295 μL Mission Bio Barcoding Mix and loaded into PCR reagent well (reservoir 8) of the DNA cartridge. The droplets from previous step (˜80 μL), 200 μL of V2 barcoding beads, and 1.25 mL of Barcoding oil were loaded into cell lysate well (reservoir 6), barcode bead well (reservoir 7) and barcode oil well (reservoir 9), respectively. The droplets were merged with barcoding beads and PCR reagents by the Cell Barcoding program. The resulting droplets were collected into the 8 PCR tubes.

In case of using custom build microfluidics, the device was first primed by filling electrode solution (2M NaCl solution) into the electrode and the moat channels. 500 μL PCR reagents containing 1.67×Q5® High-Fidelity Master Mix (NEB, M0515), 0.625 mg/mL BSA, 1.2 μM reverse primer were loaded into a 1 ml syringe. 200 μL Mission Bio V2 barcoding beads washed with Tris buffer (pH 8.0) and resuspended in 10 mM Tris buffer containing 3.75% tween 20, 2.5 mM MgCl2, 0.625 mg/mL BSA. The beads were centrifuged at 1000 RCF for 1 min and the supernatant was removed. The remaining bead slurry (˜110 μL) was loaded into PTFE tubing connected to a 1 mL syringe filled with HFE 7500 oil. The droplets after tagmentation were loaded into a 1 mL syringe. The three syringes and two syringes filled with 10 mL of 5% (w/w) PEG-PFPE surfactant (Ran Biotechnologies) in HFE 7500(3M) HFE 7500 were connected to the microfluidic devices. The follow rates are as follows: tagmentation droplets 55 μL/h, spacer oil 200 μL/h, PCR reagent 280 μL/h, barcode beads 148 μL/h, and droplet generation oil 2000 μL/h. To merge the tagmentation droplet, the electrode near the droplet generation zone was charged with an alternating current (AC) voltage (3 V, 58 kHz). And the moat channel was grounded to prevent unintended droplet coalescence at other locations on the device. The merged droplets were collected into PCR tubes.

The droplets collected in the merging step were treated with UV for 8 min (Analytik Jena Blak-Ray XX-15L UV light source) and the bottom layer of oil in each tube were removed using a gel loading tip to leave up to 100 μL of droplets. The tubes were placed on PCR instrument and thermo-cycled with the following program: 10 min at 72° C. for 1 cycle, 3 min at 95° C. for 1 cycle, (15 s at 95° C., 15 s for 55° C., and 2 min at 72° C.) for 20 cycles, and 5 min at 72° C. for 1 cycle with the lid set at 105° C.

d. Barcoded Amplicon Purification

The thermal cycled droplets in the PCR tubes were carefully transferred into two 1.5 mL centrifuge tubes (equal amount in each). If there were visible merged large droplets present, they were carefully removed using a 2 μL pipette. 20 μL PFO were added into each tube and mixed well by vortex. After centrifuging at 1000 RCF for 1 min, the top aqueous layers in each tube were transferred into new 1.5 mL tubes without disturbing the bead pellets and water was added to bring the total volume to 400 μL. The barcoding product was purified using 0.7× Ampure XP beads (Beckman Coulter, A63882) and eluted into 50 μL H2O and stored at −20° C. until next step. The concentrations of the barcoding product were measured with Qubit™ 1×dsDNA Assay Kits (ThermoFisher, Q33230).

### 5. Barcoding Sequencing Library Preparation and Sequencing

a. Library Prep and QC

The sequencing library were then prepared by attaching P5 and P7 sequences to the barcoding products using Nextera primers (Table S2). The library PCR reagents containing 25 uL Kapa HiFi Master mix 2×, 5 uL Library P5 index primer (4 uM), 5 uL Library P7 index primer (4 uM), 10 uL purified barcoding products (normalized to 0.2 ng/uL), and 5 uL of nuclease free water were thermal cycled with the following program: 3 min at 95° C. for 1 cycle, (20 s at 98° C., 20 s for 62° C., and 45 s at 72° C.) for 12 cycles, and 2 min at 72° C. for 1 cycle. The sequencing library was purified with 0.69× Ampure XP beads and eluted into 12 uL nuclease-free water. The library was quantified with Qubit™ 1×dsDNA Assay Kits and DNA HS chips on bioanalyzer or D5000 ScreenTape (Agilent, 5067-5588) on Tapestation (Agilent, G2964AA). The libraries were pooled and 300 cycle pair-end sequenced by Illumina MiSeq, NextSeq or NovaSeq platform.

### 6. Sequencing File Barcode Extraction and Single Cell Read File Preparation

Raw sequencing FASTQ files were processed using a custom python script (mb_barcode_and_trim.pys) available on GitHub (https://github.com/AbateLab/MissonBioTools) for barcode correction and extraction, adaptor trimming, and grouping by barcodes. For all reads, combinatorial cell barcodes were parsed from Read 1, using Cutadapt (v2.4) and matched to a barcode whitelist. Barcode sequences within a Hamming distance of 1 from a whitelist barcode were corrected. Reads with valid barcodes were trimmed with Cutadapt to remove 5′ and 3′ adapter sequences and demultiplexed into individual single-cell FASTQ files by barcode sequences using the script demuxbyname.sh from the BBMap package (v.38.57).

### 7. Reference Based Single Cell Data Analysis

a. ZymoBIOMICS Microbial Community Standards

The reference genome Fasta files of the ten species of Zymo BIOMICS Microbial Community Standards provided by Zymo Research Corporation (https://s3.amazonaws.com/zymo-files/BioPool/ZymoBIOMICS.STD.refseq.v2.zip). The fasta files were combined and Bowte2 index were built using Bowtie2-build command. The reads in single-cell FASTQ files were aligned to reference genomes using Bowtie2 v 2.3.5.1 with default setting. The overall alignment rates for each barcode were collected from the log files. The barcode groups less than 50% overall coverage rate were removed. Each barcode group's coverages, numbers of mapped reads, covered bases, and mean depths of 10 corresponding species were calculated using Samtools v1.12 (samtools coverage) with default setting. The purity of each barcode group was calculated as the percentage of reads that aligned to a dominant species.

b. Strain Abundance Estimation for Synthetic Community with 22 E. lenta Strains

The reference genomes of the 22 E. lenta strains were downloaded from NCBI. The reads in single-cell FASTQ files were aligned to reference genomes using Bowtie2 v 2.3.5.1 with—a setting to report all matches. The overall alignment rates for each barcode were collected from the log files. The barcode groups with less than 50% overall coverage rate were removed. The probabilities of each alignment were calculated with parseAlignment command from BitSeq v 1.16.0 using uniform read distribution option (--uniform). The abundances of the 22 strains within each barcode were calculated based on the alignment probabilities using estimate VBExpression command from BitSeq v 1.16.0 with default setting. The abundance output files were combined and analyzed using a Python script. The barcode group stain identity was assigned to the strain with maximum abundance. If the maximum abundance is smaller than 15% in a barcode group, the barcode group is considered as mixed strains. The UMAP (uniform manifold approximation and projection for dimension reduction) analysis was conducted using the Scanpy toolkit in Python.

### 8. Taxonomic Discovery Algorithm

a. TDA Validation Using Simulation Data

100 species were randomly selected from the NCBI assembly metadata file (ftp://ftp.ncbi.nlm.nih.gov/genomes/genbank/bacteria/assembly_summary.txt). The reference genome FSATA files were downloaded using the corresponding link in the metadata file. Simulated pair-end read files were generated using a Python script according to the following rules. 1. 100 barcode groups were generated for each species. 2. The reads are 150 bp pair ended. 3. The amplicon length is in the range of 400-1000 bp. 4. Each barcode group has 0-49% percent of contamination reads. 5. The contamination reads were generated from the other 99 species. 6. Each barcode has 1,000-10,000 pair-end reads.

3 taxonomic classifiers were chosen for evaluation: Kraken2/Bracken v, Kaiju v, and MetaPhlAn v. All the pair-ended barcode group FASTA files were profiled using the three classifiers. The results were grouped and analyzed in Python. The predicted taxa purity was the abundance of the dominant taxa in each barcode group. The barcode filtering based on purity was performed using thresholds ranging from 50% to 99% purities.

The average after-filtering purity was the mean purity of all the barcodes that passed a certain threshold and after-filtering barcode counts was the barcode count of which passed a certain threshold. The UMAP clustering was performed with the genus abundances of all the barcode groups. The identity of each cluster was assigned with the most abundant taxa. The identification accuracy was calculated as the percentage of barcodes with the correct genus identification.

b. TDA Analysis of Single Cell Sequence Data

The single cell sequencing barcode group FASTQ files of ZymoBIOMICS, Human microbiome and the sea water microbiome samples were analyzed using TDA with Kraken2/Bracken as the taxonomic identifier. For the ZymoBIOMICS sample, Kraken2 PlusPF database was used, while for human microbiome and sea water microbiome, Kraken2 GTDB database was used. The reads in each barcode group were first classified by Kraken2, and the abundances at genus and species level were re-estimated with Bracken using default threshold setting. The percentages of the mapped reads were extracted from the Kraken2 output files of barcode groups. The purities were calculated as the abundance of the dominant genus in the barcode groups. The data was filtered according to percentage of mapped reads and genus-level purity. The taxa abundance profiles of the remaining barcodes were combined and UMAP clustering was performed using The Scanpy toolkits in Python script. The taxa of each barcode group were assigned to the most abundant one.

### 9. Metagenomic Sequencing and Assembly

a. ZymoBIOMICS Community

The metagenomic sequencing data of ZymoBIOMICS Microbial Community Standards D6300 (batch ZRC195925) was provided by Zymo Research Corporation. The reads were assembled using SPAdes-3.15.3 with ‘--meta’ setting. The quality of assembly was accessed by Quast 5.0.2.

b. Human Microbiome

Need Cecilia's input about the metagenomic sequencing and assembly.

### 10. Comparison Between Metagenomic and Single Cell Sequencing.

The genus abundances of the human microbiome metagenomic data and the unsplitted single cell sequence file were analyzed using Kraken2 and Bracken. The results were plotted as a scatter plot with triangle markers. If any genus has a barcode group associated, a round marker of the genus was added and its size is proportional to the barcode counts.

11. Single Cell Sequencing Data Integration with Metagenomics

To integrate the metagenomic dataset, the contigs assembled from metagenomic sequencing (ZymoBIOMICS and human microbiome sample) were treated as individual barcodes and processed with TAD. The metagenomic reads were first aligned to the assembled contigs using Bowtie2 v2.3.5.1. The pair-end reads associated with each contig were extracted using Samtools v1.12 (‘samtools view -b {BAM file} {Contig header}|samtools fasta>{Extracted_reads.fa}’). The short reads from each contig were then evaluated by Kraken2 and the genus abundance were generated by Bracken using default threshold setting. The purity was calculated as the abundance of the dominant genus in each contig associated with short reads. The contigs were filtered using the genus level purity. The taxa abundance profiles of the short reads associated with remaining contigs were combined and integrated with the single cell dataset using the Scanpy toolkits in Python script.

### 12. Clustered Barcode Groups Analysis

a. Cluster Assembly and Evaluation

Single cell barcodes of UMAP clusters were combined using concatenate command (cat) in the Linux system into single FASTQ files. The pair-end reads associated with barcodes that belong to the same UMAP clusters were grouped by Seqtk toolkit (https://github.com/lh3/seqtk) (seqtk subseq) into single FASTQ files. The assemblies were conducted with all reads associated to both single cell sequencing and metagenomic contigs of each UMAP cluster using Spades v 3.15.3 with ‘--careful’ setting. The assembled contigs were evaluated using Quast v 5.0.2 with or without reference genome input. To calculate the clustering error rate, all the reads associated to a cluster were mapped to the corresponding reference genome, the percentage of the reads that were not aligned was considered as the error rate.

Pathway analyses of each cluster was conducted using HUMAnN v 3.0 with the default MetaCyc database. The pathway abundance files of each cluster were combined and plotted as a heatmap using the Seaborn module in Python.

The sub-categorizing of barcode groups in a UMAP cluster was using species abundance estimation. The 3 clusters with the most barcode groups in the human microbiome samples (Blautia_A, Bifidobacterium, and Collinsella) were further divided into sub clusters by UMAP aggregation with the Kraken2 species abundance estimation.

b. Gene Association Analysis

Comprehensive Antibiotic Resistance Database CARD v 3.1.4 (https://card.mcmaster.ca/download), Virulence factor database v 10.06.01 (http://www.mgc.ac.cn/VFs/), Plasmid database v 2020 Nov. 19 (https://ccb-microbe.cs.uni-saarland.de/plsdb/), and Phage gene database v 1 Nov. 2021 (https://github.com/RyanCook94/inphared) were downloaded and bowtie2 references were build.

The combined reads associated with each UMAP cluster identified in the sea water sample were mapped to the 4 databases using Bowtie2. The mapping reads are filtered for MAPQ>2 to remove ambiguously aligned reads (samtools view -bS -q 2). After duplication removal, the references sequence name (RNAME) of each alignment were extracted from the bam files. The unique genes associated with each UMAP cluster, and their frequencies were generated from the RNAMEs. To generate the antibiotic resistant gene association heatmap, the antibiotic resistant ontology accessions (ARO) were obtained from the RNAMEs of each alignment. The drug class associated with AROs were downloaded from the Comprehensive Antibiotic Resistance Database. The heatmap intensities were calculated as the total read counts associated with a specific drug class normalized to the total barcode group count within a given UMAP cluster. To generate the heatmap for transduction potential, phage ID were extracted from the RNAMEs of each alignment. The heatmap intensities were calculated as follows: for a given pair of UMAP clusters, the total read counts of all shared phages were normalized to the total counts of the barcode groups of the two clusters.

### REFERENCES

1. Cho, I. & Blaser, M. J. The human microbiome: at the interface of health and disease. Nature Reviews Genetics 13, 260-270 (2012).
2. Weersma, R. K., Zhernakova, A. & Fu, J. Interaction between drugs and the gut microbiome. Gut 69, 1510-1519 (2020).
3. Singh, B. K., Trivedi, P., Egidi, E., Macdonald, C. A. & Delgado-Baquerizo, M. Crop microbiome and sustainable agriculture. Nature Reviews Microbiology 18, 601-602 (2020).
4. Jørgensen, B. B. & Boetius, A. Feast and famine—microbial life in the deep-sea bed. Nature Reviews Microbiology 5, 770-781 (2007).
5. FAQ: Microbes and Climate Change. (2017) doi:10.1128/AAMCol.Mar.2016.
6. Muir, P. et al. The real cost of sequencing: scaling computation to keep pace with data generation. Genome Biology 17, 53 (2016).
7. Porter, T. M. & Hajibabaei, M. Scaling up: A guide to high-throughput genomic approaches for biodiversity analysis. Molecular Ecology 27, 313-338 (2018).
8. Pérez-Cobas, A. E., Gomez-Valero, L. & Buchrieser, C. Metagenomic approaches in microbial ecology: an update on whole-genome and marker gene sequencing analyses. Microbial Genomics 6, (2020).
9. Caporaso, J. G. et al. Global patterns of 16S rRNA diversity at a depth of millions of sequences per sample. Proceedings of the National Academy of Sciences 108, 4516-4522 (2011).
10. Schoch, C. L. et al. Nuclear ribosomal internal transcribed spacer (ITS) region as a universal DNA barcode marker for Fungi. Proceedings of the National Academy of Sciences 109, 6241-6246 (2012).
11. Tyson, G. W. et al. Community structure and metabolism through reconstruction of microbial genomes from the environment. Nature 428, 37-43 (2004).
12. Quince, C., Walker, A. W., Simpson, J. T., Loman, N. J. & Segata, N. Shotgun metagenomics, from sampling to analysis. Nature Biotechnology 35, 833-844 (2017).
13. Bharti, R. & Grimm, D. G. Current challenges and best-practice protocols for microbiome analysis. Briefings in Bioinformatics 22, 178-193 (2021).
14. Liao, X. et al. Current challenges and solutions of de novo assembly. Quantitative Biology 7, 90-109 (2019).
15. Wooley, J. C. & Ye, Y. Metagenomics: Facts and Artifacts, and Computational Challenges. Journal of Computer Science and Technology 25, 71-81 (2010).
16. Keloth, A., Anderson, O., Risbridger, D. & Paterson, L. Single Cell Isolation Using Optical Tweezers. Micromachines (Basel) 9, (2018).
17. Zhang, H. & Liu, K.-K. Optical tweezers for single cells. Journal of The Royal Society Interface 5, 671-690 (2008).
18. Bowers, R. M. et al. Dissecting the dominant hot spring microbial populations based on community-wide sampling at single-cell genomic resolution. The ISME Journal 16, 1337-1347 (2022).
19. Rinke, C. et al. Obtaining genomes from uncultivated environmental microorganisms using FACS-based single-cell genomics. Nature Protocols 9, 1038-1048 (2014).
20. Tamminen, M. v. & Virta, M. P. J. Single gene-based distinction of individual microbial genomes from a mixed population of microbial cells. Frontiers in Microbiology 6, (2015).
21. Xu, L., Brito, I. L., Alm, E. J. & Blainey, P. C. Virtual microfluidics for digital quantification and single-cell sequencing. Nature Methods 13, 759-762 (2016).
22. Podar, M. et al. Targeted Access to the Genomes of Low-Abundance Organisms in Complex Microbial Communities. Applied and Environmental Microbiology 73, 3205-3214 (2007).
23. Leung, K. et al. Robust high-performance nanoliter-volume single-cell multiple displacement amplification on planar substrates. Proceedings of the National Academy of Sciences 113, 8484-8489 (2016).
24. Gawad, C., Koh, W. & Quake, S. R. Dissecting the clonal origins of childhood acute lymphoblastic leukemia by single-cell genomics. Proc Natl Acad Sci USA 111, 17947-17952 (2014).
25. Chijiiwa, R. et al. Single-cell genomics of uncultured bacteria reveals dietary fiber responders in the mouse gut microbiota. Microbiome 8, 5 (2020).
26. Nishikawa, Y. et al. Massively parallel single-cell genome sequencing enables high-resolution analysis of soil and marine microbiome. bioRxiv 2020.03.05.962001 (2020) doi: 10.1101/2020.03.05.962001.
27. Kuchina, A. et al. Microbial single-cell RNA sequencing by split-pool barcoding. Science (1979) 371, eaba5257 (2021).
28. Lan, F., Demaree, B., Ahmed, N. & Abate, A. R. Single-cell genome sequencing at ultra-high-throughput with microfluidic droplet barcoding. Nature Biotechnology 35, 640-646 (2017).
29. Zheng, W. et al. Microbe-seq: high-throughput, single-microbe genomics with strain resolution, applied to a human gut microbiome. bioRxiv 2020.12.14.422699 (2020).
30. de Bruin, O. M. & Birnboim, H. C. A method for assessing efficiency of bacterial cell disruption and DNA release. BMC Microbiology 16, 197 (2016).
31. Wood, D. E., Lu, J. & Langmead, B. Improved metagenomic analysis with Kraken 2. Genome Biology 20, 257 (2019).
32. Lu, J., Breitwieser, F. P., Thielen, P. & Salzberg, S. L. Bracken: estimating species abundance in metagenomics data. Peer J Computer Science 3, e104 (2017).
33. Beghini, F. et al. Integrating taxonomic, functional, and strain-level profiling of diverse microbial communities with bioBakery 3. Elife 10, (2021).
34. Menzel, P., Ng, K. L. & Krogh, A. Fast and sensitive taxonomic classification for metagenomics with Kaiju. Nature Communications 7, 11257 (2016).
35. Bisanz, J. E. et al. A Genomic Toolkit for the Mechanistic Dissection of Intractable Human Gut Bacteria. Cell Host & Microbe 27, 1001-1013.e9 (2020).
36. Glaus, P., Honkela, A. & Rattray, M. Identifying differentially expressed transcripts from RNA-seq data with biological variation. Bioinformatics 28, 1721-1728 (2012).
37. Hevia, A., Delgado, S., Margolles, A. & Sánchez, B. Application of density gradient for the isolation of the fecal microbial stool component and the potential use thereof. Scientific Reports 5, 16807 (2015).
38. Bäckhed, F., Ley, R. E., Sonnenburg, J. L., Peterson, D. A. & Gordon, J. I. Host-Bacterial Mutualism in the Human Intestine. Science (1979) 307, 1915-1920 (2005).
39. Watson, E.-J., Giles, J., Scherer, B. L. & Blatchford, P. Human faecal collection methods demonstrate a bias in microbiome composition by cell wall structure. Scientific Reports 9, 16831 (2019).
40. Franzosa, E. A. et al. Species-level functional profiling of metagenomes and metatranscriptomes. Nature Methods 15, 962-968 (2018).
41. Caspi, R. et al. The MetaCyc database of metabolic pathways and enzymes and the BioCyc collection of Pathway/Genome Databases. Nucleic Acids Research 42, D459-D471 (2014).
42. Han, J.-R., Ye, M.-Q., Wang, C. & Du, Z.-J. Halioglobus sediminis sp. nov., isolated from coastal sediment. International Journal of Systematic and Evolutionary Microbiology 69, 1601-1605 (2019).
43. Ochman, H. & Moran, N. A. Genes Lost and Genes Found: Evolution of Bacterial Pathogenesis and Symbiosis. Science (1979) 292, 1096-1099 (2001).
44. Tang, F. et al. mRNA-Seq whole-transcriptome analysis of a single cell. Nature Methods 6, 377-382 (2009).
45. Hwang, B., Lee, J. H. & Bang, D. Single-cell RNA sequencing technologies and bioinformatics pipelines. Experimental & Molecular Medicine 50, 1-14 (2018).
46. Stoeckius, M. et al. Simultaneous epitope and transcriptome measurement in single cells. Nature Methods 14, 865-868 (2017).
47. Demaree, B. et al. Joint profiling of DNA and proteins in single cells to dissect genotype-phenotype associations in leukemia. Nature Communications 12, 1583 (2021).
48. Ståhl, P. L. et al. Visualization and analysis of gene expression in tissue sections by spatial transcriptomics. Science (1979) 353, 78-82 (2016).
49. Stuart, T. & Satija, R. Integrative single-cell analysis. Nature Reviews Genetics 20, 257-272 (2019).
50. Tan, L., Xing, D., Chang, C.-H., Li, H. & Xie, X. S. Three-dimensional genome structures of single diploid human cells. Science (1979) 361, 924-928 (2018).
51. Mulqueen, R. M. et al. High-content single-cell combinatorial indexing. Nature Biotechnology 39, 1574-1580 (2021).
52. Sheth, R. U. et al. Spatial metagenomic characterization of microbial biogeography in the gut. Nature Biotechnology 37, 877-883 (2019).

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of sequencing one or more nucleic acids from a single cell comprising the steps of:
(a) preparing single cells for coding comprising compartmentalizing a population of cells in permeable compartments comprising single cells;
(b) barcoding nucleic acid molecules associated with the single cells, wherein a unique barcode is used for each single cell; and
(c) sequencing one or more nucleic acids from a single cell.

2. The method of claim 1, wherein the compartmentalizing in step (a) comprises: (i) encapsulating single cells into hydrogel particles or (ii) fixating cells under conditions that allow formation of permeable, single-cell particles.

3. The method of claim 2, wherein the compartmentalizing comprises encapsulating single cells into hydrogel particles.

4. The method of claim 3, wherein the preparing single cells for barcoding comprises:
(a) preparing a suspension comprising a population of single cells;
(b) encapsulating the population of single cells in a polydispersed emulsion comprising gel droplets to provide a population of gel droplets, wherein each gel droplet contains zero cells, a single cell, or multiple cells;
(c) polymerizing the population of gel droplets to provide a population of polymerized gel droplets;
(d) separating the polymerized gel droplets by size under conditions that allow selection of a population of gel droplets that each comprise a single cell;
(e) optionally amplifying one or more biomolecules under conditions to increase copy number of said one or more biomolecules; and
(f) lysing the single cells within the population of gel droplets under conditions that allow cell lysis.

5. The method of claim 4, wherein the steps of preparing a suspension in step (a) and encapsulating in step (b) are not performed using a microfluidic device.

6. The method of claim 5, wherein one or more of the steps of polymerizing in step (c), separating in step (d), optionally the amplifying in step (e), lysing in step (f), and barcoding are optionally not performed using a microfluidic device.

7. The method of claim 4 wherein the preparing comprises the amplifying of step (e), and wherein said amplifying comprises an amplification technique selected from the group consisting of multiple displacement amplification (MDA), looping-based amplification cycles (MALBAC), degenerate oligonucleotide PCR (DOP-PCR) and primer extension pre-amplification (PEP).

8. The method of claim 4, wherein step (e) occurs before step (d).

9. The method of claim 4, wherein the suspension comprising the population of cells comprises an unpolymerized monomer solution.

10. The method of claim 9, wherein the unpolymerized monomer is selected from the group consisting of acrylamide, N,N′-Bis(acryloyl)cystamine (BAC), Bis(2-methacryloyl)oxyethyl disulfide (DSDMA), N,N′-(1,2-Dihydroxyethylene)bisacrylamide (DHEBA), N,N′-Methylene-bis-acrylamide or agarose.

11. The method of claim 9, wherein the suspension further comprises a stain, antibody, aptamer, label, or affinity reagent capable of binding to one or more biomolecules including nucleic acids prior to and/or following cell lysis in step (e).

12. The method of claim 11, wherein the stain is capable of binding to a cell membrane or cell wall.

13. The method of claim 4, wherein the encapsulating the population of cells of step (b) comprises adding an immiscible carrier and agitating under conditions that allow formation of a polydispersed emulsion.

14. The method of claim 13, wherein the agitating is selected from the group consisting of pipetting, shaking by hand, stirring, beating, bubbling, passing the solution through a needle, vortexing and sonicating.

15. The method of claim 4, wherein the gel droplets are the approximate size of a mammalian cell.

16. The method of any of claim 4, wherein the polymerizing of step (c) comprises one or more of cooling, chemical crosslinking, photo-crosslinking, and ionic interaction crosslinking.

17. The method of any of claim 4, wherein the separating of step (d) comprises one or more of centrifugation, density centrifugation, filtration, and/or collecting layers.

18. The method of any of claim 4, wherein the lysing step (e) comprises adding a solution comprising a detergent and/or an enzyme and/or heating.

19. The method of claim 18 wherein the detergent is selected from the group consisting of sodium dodecyl sulfate (SDS), Tween, Triton, Brij, Octyl glucoside, 3-[(3-Cholamidopropyl)dimethylammonio]-1-propanesulfonate (CHAPS), and 3-([3-Cholamidopropyl]dimethylammonio)-2-hydroxy-1-propanesulfonate (SHAPSO).

20. The method of claim 18, wherein the enzyme is selected from the group consisting of lysozyme, proteinase K, lysostaphin, zymolyase, and mutanolysin.

21. The method of claim 4, wherein the lysing step (e) further comprises processing one or more biomolecules from the lysed cells, wherein said biomolecules comprise nucleic acids and/or proteins, and wherein said processing comprises digesting, labelling, capturing, and/or conjugating said biomolecules.

22. The method of claim 21, wherein one or more biomolecules are labeled with a fluorophore or antibody.

23. The method of claim 21, wherein one or more biomolecules are digested.

24. The method of claim 2, wherein the compartmentalizing comprises fixating cells under conditions that allow formation of permeable, single-cell particles.

25. The method of claim 24, wherein the preparing single cells for barcoding comprises:
(a) preparing a suspension comprising a population of single cells;
(b) fixating single cells under conditions that allow formation of permeable, single-cell particles;
(c) separating the single-cell particles; and
(d) lysing the single cells within the single-cell particles under conditions that allow cell lysis.

26. The method of claim 25, wherein the steps of preparing a suspension in step (a) and fixating in step (b) are not performed using a microfluidic device.

27. The method of claim 26, wherein one or more of the separating in step (c), the lysing in step (d), and barcoding are optionally not performed using a microfluidic device.

28. The method of claim 25, wherein step (d) occurs before step (c).

29. The method of claim 25, wherein the suspension further comprises a stain, antibody, aptamer, label, or affinity reagent capable of binding to one or more biomolecules including nucleic acids prior to and/or following cell lysis in step (d).

30. The method of claim 29, wherein the stain is capable of binding to a cell membrane or cell wall.

31. The method of claim 25, wherein the fixating single cells of step (b) comprises adding one or more fixation reagents selected from the group consisting of an organic solvent, a crosslinked fixative, or combinations thereof, under conditions that formation of permeable, single-cell particles.

32. The method of claim 31, wherein the organic solvent is selected from the group consisting of methanol, ethanol, acetic acid, or combinations thereof.

33. The method of claim 31, wherein the crosslinked fixative is selected from the group consisting of formaldehyde, paraformaldehyde, glutaraldehyde, acrolein, dithio-bis(succinimidyl propionate); oxidants: osmium tetroxide, potassium dichromate, or combinations thereof.

34. The method of claim 25, wherein the separating of step (c) comprises one or more of centrifugation, density centrifugation, filtration, and/or collecting layers.

35. The method of any of claim 25, wherein the lysing step (d) comprises adding a solution comprising a detergent and/or an enzyme and/or heating.

36. The method of claim 35, wherein the detergent is selected from the group consisting of sodium dodecyl sulfate (SDS), Tween, Triton, Brij, Octyl glucoside, 3-[(3-Cholamidopropyl)dimethylammonio]-1-propanesulfonate (CHAPS), and 3-([3-Cholamidopropyl]dimethylammonio)-2-hydroxy-1-propanesulfonate (SHAPSO).

37. The method of claim 35, wherein the enzyme is selected from the group consisting of lysozyme, proteinase K, lysostaphin, zymolyase, and mutanolysin.

38. The method of claim 25, wherein the lysing step (e) further comprises processing one or more biomolecules from the lysed cells, wherein said biomolecules comprise nucleic acids and/or proteins, and wherein said processing comprises digesting, labelling, capturing, and/or conjugating said biomolecules.

39. The method of claim 38, wherein one or more biomolecules are labeled with a fluorophore or antibody.

40. The method of claim 38, wherein one or more biomolecules are digested.

41. The method of any of the preceding claims, wherein cells are selected from mammalian cells, bacterial cells, fungal cells, yeast cells, and plant cells.

42. The method of claim 41, wherein the cells are bacterial cells.

43. The method of claim 41, wherein the cells are human cells taken from a saliva, blood, urine, or tissue sample.

44. The method of any of the preceding claims, wherein the one or more nucleic acids are selected from the group consisting of DNA, genomic DNA, RNA and mRNA.

45. A method of determining the presence of a biomolecule associated with a single cell comprising the steps of preparing single cells for barcoding according to claim 4 or claim 24, wherein one or more nucleic acids are conjugated to a reagent capable of specifically binding to the biomolecule.
